(19) United States
(12) Patent Application Publication
(10) Pub. No.: US 2023/0218571 A1
(43) Pub. Date: Jul. 13, 2023

(54) CYSTEAMINE PRECURSOR COMPOUNDS FOR THE TREATMENT OF BETACORONAVIRUS INFECTIONS

(71) Applicant: Thiogenesis Therapeutics, Inc., San Diego, CA (US)

(72) Inventor: Patrice P. RIOUX, San Diego, CA (US)

(21) Appl. No.: 17/916,212

(22) PCT Filed: Mar. 31, 2021
(86) PCT No.: PCT/US2021/025070

§ 371 Date: Sep. 30, 2022

Related U.S. Application Data
(60) Provisional application No. 63/021,180, filed on May 7, 2020.
(60) Provisional application No. 63/003,429, filed on Apr. 1, 2020.

(51) Int. Cl. A61K 31/385; A61P 31/14

(52) U.S. Cl. CPC A61K 31/385; A61P 31/14

(57) ABSTRACT

The invention features the use of cysteamine precursor compounds for the treatment and prophylaxis of severe symptoms of betacoronavirus infections, such as infections by SARS-CoV-2, SARS-CoV-1, MERS-CoV, and related viruses.

### BACKGROUND OF THE INVENTION

During the first weeks of 2020, the world has evidenced the emergence of a new human pathogen that achieved enough zoonotic spillover to cause a pandemic, from a highly pathogenic betacoronavirus.

The 2019 novel Coronavirus (SARS-CoV-2) that is the cause of the highly infectious disease known as COVID-19, is a new member of a group, that includes previously recognized zoonotic pathogens, as is the case of the Severe Acute Respiratory Syndrome Coronavirus (SARS-CoV-1), that caused epidemics in China in 2002-2003, and the Middle East Respiratory Syndrome (MERS-CoV), affecting Saudi Arabia and neighbor countries in 2012-2013.

Based on hospitalized patient data, the majority of COVID-19 cases (about 80%) present with asymptomatic or mild symptoms while the remainder are severe or critical (Huang et al., Lancet 395:497 (2020); Chan et al., Lancet 395:514 (2020)). It seems that the severity and fatality rate of COVID-19 is milder than that of SARS and MERS, but infectivity is greater. With similar clinical presentations as SARS and MERS, the most common symptoms of COVID-19 are fever, fatigue, and respiratory symptoms, including cough, sore throat and shortness of breath. A study of 41 hospitalized patients showed high-levels of proinflammatory cytokines were observed in the COVID-19 severe cases (Huang et al., Lancet 395:497 (2020). These findings are in line with SARS and MERS in that the presence of lymphopenia and “cytokine storm” likely plays a major role in the pathogenesis of COVID-19 (see, e.g., Nicholls et al., Lancet.; 361(9371):1773 (2003); Mahallawi et al., Cytokine.; 104:8 (2018); and Wong et al., Clin Exp Immunol. 136(1):95 (2004)). This so-called “cytokine storm” can initiate viral sepsis and inflammatory-induced lung injury which can lead to other complications, including pneumonitis, pneumonia, acute respiratory distress syndrome (ARDS), pneumonia, respiratory failure, septic shock, organ failure and death.

COVID-19 emerged recently in China and quickly has spread worldwide, resulting in >854,039 confirmed cases and 42,014 deaths as of Mar. 31, 2020. There is an urgent need for safe and effective therapeutic and prophylactic agents against betacoronavirus infections, such as infections by SARS-CoV-2, SARS-CoV-1, MERS-CoV, and related viruses.

### SUMMARY OF THE INVENTION

The invention features the use of cysteamine precursor compounds for the treatment and prophylaxis of symptoms of beta-coronavirus infections, such as infections by SARS-CoV-2, SARS-CoV-1, MERS-CoV, and related viruses.

In a first aspect, the invention features a method of treating a betacoronavirus infection in a human subject, the method including administering to the subject a therapeutically effective amount of a cysteamine precursor compound or a pharmaceutically acceptable salt thereof.

The invention further features a method of ameliorating one or more symptoms of a betacoronavirus infection in a human subject, the method including administering to the subject a therapeutically effective amount of a cysteamine precursor compound or a pharmaceutically acceptable salt thereof. The one or more symptoms can include fever, cough, shortness of breath, bilateral lung involvement with ground-glass opacity (observable from computed tomography images), or any other symptom described herein. The one or more symptoms can be reduced either in their frequency by 10%, 20%, 30%, or 50% relative to control subjects of the same age and having the same comorbidities as untreated.

The invention also features a method of inhibiting the progression of a betacoronavirus infection in a human subject, the method including administering to the subject a therapeutically effective amount of a cysteamine precursor compound or a pharmaceutically acceptable salt thereof. For example, the risk of progression to pneumonitis, pneumonia, acute respiratory distress syndrome, respiratory failure, septic shock, organ failure, cytokine storm, and/or death can be inhibited by 10%, 20%, 30%, or 50% relative to control subjects of the same age and having the same comorbidities as untreated.

In a related aspect, the invention features a method of reducing the likelihood of betacoronavirus infection in a human subject at risk thereof, the method including administering to the subject a therapeutically effective amount of a cysteamine precursor compound or a pharmaceutically acceptable salt thereof. The subject at risk can be a subject know to have been in contact with an infected person or in contact with a location previously occupied by an infected person. In certain embodiments, the subject at risk is under quarantine. The likelihood of betacoronavirus infection can be reduced by 10%, 20%, 30%, or 50% relative to control subjects of the same age and having the same comorbidities, as untreated control subjects.

In particular embodiments of any of the above methods, the risk of hospitalization of the subject is reduced. In other embodiments of any of the above methods, the duration of hospitalization is reduced.

In embodiments of any of the above methods, the administration occurs between once per week to three times per day. For example, the administration can be once per day or twice per day. The administration can occur over a treatment period, e.g., of about 1 day to about 21 days (e.g., 1 to 14 days, 7±3 days, 10±4 days, 15±6 days), or from about 1 week to about 6 weeks, or over a longer treatment period, if necessary.

In particular embodiments of the above methods, the subject is being hospitalized or quarantined for the betacoronavirus infection.

In some embodiments of any of the above methods, the subject has a pre-existing condition that places the subject at higher risk of pneumonitis, pneumonia, acute respiratory distress syndrome, pneumonia, respiratory failure, septic shock, organ failure, cytokine storm, or death. For example, the subject at higher risk can be one having a pre-existing condition selected from cardiovascular disease, diabetes, chronic respiratory disease, hypertension, and obesity.

In other embodiments of any of the above methods, the subject is at least 20, 30, 40, 50, 60, 70, or 80 years old.

In one embodiment of any of the above methods, the betacoronavirus is SARS-CoV-2.

In another embodiment of any of the above methods, the betacoronavirus is SARS-CoV-1.

In still another embodiment of any of the above methods, the betacoronavirus is MERS-CoV.

In still another embodiment of any of the above methods, the betacoronavirus is a mutated form of SARS-CoV-1 or SARS-CoV-2.

In some embodiments of any of the above methods, the cysteamine precursor is selected from pantetheine-N-acetyl-L-cysteine disulfide, pantetheine-N-acetylcysteamine disulfide, cysteamine-pantetheine disulfide, cysteamine-4-phosphopantetheine disulfide, cysteamine-gamma-glutamylcysteine disulfide or cysteamine-N-acetylcysteine disulfide, mono-cysteamine-dihydrolipoic acid disulfide, bis-cysteamine-dihydrolipoic acid disulfide, mono-pantetheine-dihydrolipoic acid disulfide, bis-pantetheine-dihydrolipoic acid disulfide, cysteamine-pantetheine-dihydrolipoic acid disulfide, and salts thereof.

In particular embodiments of any of the above methods, the cysteamine precursor is selected from compounds (1)-(3):

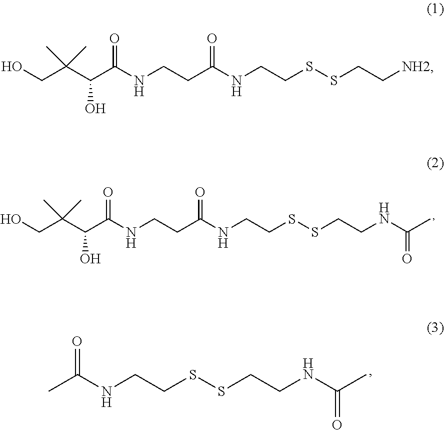

and salts thereof.

### Definitions

As used herein, the term “about” means +/−10% of the recited value.

As used herein, by “administration” or “administering” is meant a method of giving a dosage of a cysteamine precursor compound to a subject. The cysteamine precursor compounds utilized in the methods described herein can be administered, for example, orally, or by another other route described herein.

By “cysteamine precursor” is meant a compound that can be converted under physiological conditions into at least one cysteamine. The means of conversion include reduction in the case of cysteamine containing disulfides (i.e. cysteamine mixed disulfides), enzymatic hydrolysis in the case of pantetheinase substrates (pantetheine as well as compounds that are metabolically convertible into pantetheine in the gastrointestinal tract, such as 4-phosphopantetheine, dephospho-coenzyme A and coenzyme A and suitable analogs or derivatives thereof, or both reduction and enzymatic cleavage. Examples of precursors include, but are not limited to, cysteamine mixed disulfides, pantetheine disulfides, 4-phosphopantetheine disulfides, dephospho-coenzyme A disulfides, coenzyme A disulfides and N-acetylcysteamine disulfides, as well as pantetheine, 4-phosphopantetheine, dephospho-coenzyme A, coenzyme A, and N-acetylcysteamine. The chemical relationship between cysteamine, pantetheine, 4-phosphopantetheine, dephospho-coenzyme A and coenzyme A (the four latter compounds being cysteamine precursors) is illustrated as follows. A homodimer of two pantetheine molecules (i.e. pantethine), or of two 4-phosphopantetheine molecules, or of two dephospho-coenzyme A molecules or of two coenzyme A molecules or of two N-acetylcysteamine molecules are also each disulfide cysteamine precursor compounds, as the constituent thiols are all cysteamine precursors.

As used herein, a “therapeutically-effective amount” refers to that amount that must be administered to a patient (a human or non-human mammal) in order to ameliorate a symptom of COVID-19 in a subject.

As used herein, “reducing the risk of pneumonitis” in a subject refers to reducing the frequency of pneumonitis in subjects treated according to the methods of the invention. The reduction is in comparison to control subjects of the same age and condition (e.g., comorbidities) that are untreated. The frequency of pneumonitis can be reduced by 10%, 20%, 30%, or 50% relative to the frequency of pneumonitis observed for the control subjects.

As used herein, “reducing the risk of acute respiratory distress syndrome” in a subject refers to reducing the frequency of acute respiratory distress syndrome in subjects treated according to the methods of the invention. The reduction is in comparison to control subjects of the same age and condition (e.g., comorbidities) that are untreated. The frequency of acute respiratory distress syndrome can be reduced by 10%, 20%, 30%, or 50% relative to the frequency of acute respiratory distress syndrome observed for the control subjects.

As used herein, “reducing the risk of respiratory failure” in a subject refers to reducing the frequency of respiratory failure in subjects treated according to the methods of the invention. The reduction is in comparison to control subjects of the same age and condition (e.g., comorbidities) that are untreated. The frequency of respiratory failure can be reduced by 10%, 20%, 30%, or 50% relative to the frequency of respiratory failure observed for the control subjects.

As used herein, “reducing the risk of pneumonia” in a subject refers to reducing the frequency or severity of pneumonia in subjects treated according to the methods of the invention. The reduction is in comparison to control subjects of the same age and condition (e.g., comorbidities) that are untreated. The frequency or severity of pneumonia can be reduced by 10%, 20%, 30%, or 50% relative to the frequency or severity of pneumonia observed for the control subjects.

As used herein, “reducing the risk of septic shock” in a subject refers to reducing the frequency of septic shock in subjects treated according to the methods of the invention. The reduction is in comparison to control subjects of the same age and condition (e.g., comorbidities) that are untreated. The frequency of septic shock can be reduced by 10%, 20%, 30%, or 50% relative to the frequency of septic shock observed for the control subjects.

As used herein, “reducing the risk of organ failure” in a subject refers to reducing the frequency of organ failure in subjects treated according to the methods of the invention. The reduction is in comparison to control subjects of the same age and condition (e.g., comorbidities) that are untreated. The frequency of organ failure can be reduced by 10%, 20%, 30%, or 50% relative to the frequency of organ failure observed for the control subjects.

As used herein, “reducing the risk of death” in a subject refers to reducing the frequency of death in subjects treated according to the methods of the invention. The reduction is in comparison to control subjects of the same age and condition (e.g., comorbidities) that are untreated. The frequency of death can be reduced by 10%, 20%, 30%, or 50% relative to the frequency of death observed for the control subjects.

As used herein, “reducing the risk of cytokine storm” in a subject refers to reducing the frequency of cytokine storm in subjects treated according to the methods of the invention. The reduction is in comparison to control subjects of the same age and condition (e.g., comorbidities) that are untreated. The frequency of cytokine storm can be reduced by 10%, 20%, 30%, or 50% relative to the frequency of cytokine storm observed for the control subjects.

As used herein, “reducing the risk of hospitalization” in a subject refers to reducing the frequency of hospitalization in subjects treated according to the methods of the invention. The reduction is in comparison to control subjects of the same age and condition (e.g., comorbidities) that are untreated. The frequency of hospitalization can be reduced by 10%, 20%, 30%, or 50% relative to the frequency of hospitalization observed for the control subjects.

As used herein, “reducing the duration of hospitalization” in a subject refers to reducing the duration of hospitalization in subjects treated according to the methods of the invention. The reduction is in comparison to control subjects of the same age and condition (e.g., comorbidities) that are untreated. The duration of hospitalization can be reduced by 10%, 20%, 30%, or 50% relative to the duration of hospitalization observed for the control subjects.

As used herein, a “therapeutically effective amount” refers to an amount of a cysteamine precursor compound required to treat, ameliorate the symptoms of, inhibit the progression of, or reduce the likelihood of developing a betacoronavirus infection. The effective amount of a cysteamine precursor compound used to practice the invention for therapeutic or prophylactic treatment of conditions caused by or contributed to by a betacoronavirus infection varies depending upon the manner of administration, the age, body weight, and general health of the subject. Ultimately, the attending physician will decide the appropriate amount and dosage regimen. Such amount is referred to as a “therapeutically effective amount.”

By “pharmaceutical composition” is meant any composition that contains a cysteamine precursor compound combined with a pharmaceutically acceptable carrier that together is suitable for administration to a subject and that treats or prevents a betacoronavirus infection or reduces the severity of, or ameliorates, one or more symptoms associated with a betacoronavirus infection Pharmaceutical compositions useful in the methods of the invention can take the form of tablets, gelcaps, capsules, pills, powders, granulates, suspensions, and/or emulsions.

As used herein, the term “pharmaceutically acceptable carrier” refers to an excipient or diluent in a pharmaceutical composition. For example, a pharmaceutically acceptable carrier may be a vehicle capable of suspending or dissolving the active ingredients (e.g., a cysteamine precursor compound). The pharmaceutically acceptable carrier can be compatible with the other ingredients of the formulation and not deleterious to the recipient. For oral administration, a solid carrier may be preferred.

As used herein, the term “treat” or “treating” includes administration of a cysteamine precursor compound to a subject by any route, e.g., orally. The subject, e.g., a patient, can be one having a disorder (e.g., a disease or condition described herein), a symptom of a disorder, or a predisposition toward a disorder. Treatment is not limited to curing or complete healing, but can result in one or more of alleviating, relieving, altering, partially remedying, ameliorating, improving or affecting the betacoronavirus infection, reducing one or more symptoms of the betacoronavirus infection or the predisposition toward the betacoronavirus infection. In an embodiment the treatment (at least partially) alleviates or relieves symptoms related to a betacoronavirus infection. In one embodiment, the treatment reduces at least one symptom of the betacoronavirus infection or delays onset of at least one symptom of the betacoronavirus infection. The effect is beyond what is seen in the absence of treatment.

As used herein, the term “pharmaceutically acceptable salt” refers to salt forms (e.g., acid addition salts or metal salts) of the cysteamine precursor compounds suitable for therapeutic use according to the methods of the invention.

Other features and advantages of the invention will be apparent from the following Detailed Description, and the claims.

### DETAILED DESCRIPTION OF THE INVENTION

The invention features methods for treating or preventing infections by betacoronavirus pathogens, including SARS-CoV-1 (that caused epidemics in China in 2002-2003), MERS-CoV (that affected Saudi Arabia and neighbor countries in 2012-2013), and SARS-CoV-2 (which emerged recently in China and quickly has spread worldwide). The methods include administering a cysteamine precursor compound to a subject suffering, or at risk of, the infection.

With similar clinical presentations as SARS and MERS, the most common symptoms of COVID-19 are fever, fatigue, and respiratory symptoms, including cough, sore throat and shortness of breath. Such infections are characterized by high-levels of proinflammatory cytokines resulting in a “cytokine storm” that likely plays a major role in the pathogenesis of these infections. This so-called “cytokine storm” can initiate viral sepsis and inflammatory-induced lung injury which lead to other complications including pneumonitis, pneumonia, acute respiratory distress syndrome (ARDS), respiratory failure, septic shock, organ failure and death.

Following administration, cysteamine precursors produce cysteamine in vivo, which can subsequently bind cysteine through a sulfur bond to form a cysteamine-cysteine disulfide. All major manifestations of SARS-Cov2 (acute respiratory distress syndrome, cytokine storms, clotting, congestive heart failure, viral replication) can be sensitive to treatment with cysteamine. Coronavirus-induced membrane fusion requires the cysteine-rich domain in the spike protein: site-specific mutations of conserved cysteine residues in the cys domain markedly reduce membrane fusion, which further supports the conclusion that this region is crucial for spike function (see, e.g., Chang et al., Virology 269:212-24 (2000)). Coronavirus envelope (E) proteins play an important, not fully understood role(s) in the virus life cycle. All E proteins have conserved cysteine residues located on the carboxy side of the long hydrophobic domain, suggesting functional significance (see, e.g., Lopez et al., Journal of Virology 82:3000-10 (2008)). The methods of the invention can provide one or more of the following benefits for subjects suffering from COVID-19: (i) antioxidant effect (e.g., for treatment of ARDS); (ii) inhibition of transglutaminase 2 (e.g., for treatment of cytokine release syndrome (CRS) and/or pathogenic clotting); (iii) cardioprotective effect (e.g., for treatment of congestive heart failure); and (iv) antiviral properties (e.g., for treatment of viral infection to reduce viral load and/or reduce infectiousness).

Provided herein are methods of using cysteamine precursor compounds for treating, ameliorating the symptoms of, inhibiting progression of, or reducing the likelihood of developing a betacoronavirus infection in a subject.

The invention features compositions and methods that permit treatment with cysteamine from precursor compounds (cysteamine precursors) in controlled amounts and at controlled locations in the gastrointestinal tract, and methods of treating betacoronavirus infections therewith.

In any of the methods of the invention, the cysteamine precursor can be selected from pantetheine-N-acetyl-L-cysteine disulfide, pantetheine-N-acetylcysteamine disulfide, cysteamine-pantetheine disulfide, cysteamine-4-phosphopantetheine disulfide, cysteamine-gamma-glutamylcysteine disulfide or cysteamine-N-acetylcysteine disulfide, mono-cysteamine-dihydrolipoic acid disulfide, bis-cysteamine-dihydrolipoic acid disulfide, mono-pantetheine-dihydrolipoic acid disulfide, bis-pantetheine-dihydrolipoic acid disulfide, cysteamine-pantetheine-dihydrolipoic acid disulfide, and salts thereof. For example, the methods of the invention can include any one of compounds 1-3, shown below, or a pharmaceutically acceptable salt thereof.

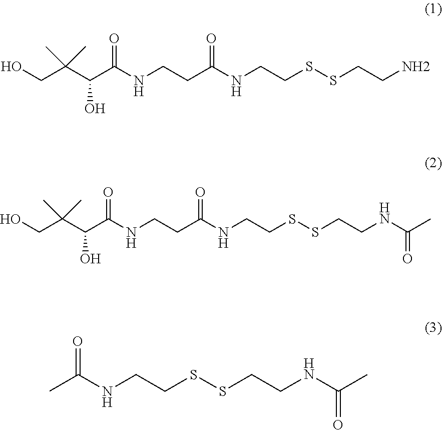

Compounds 1-3 can be administered alone, or in combination with a second active that is a cysteamine precursor, or in combination with an agent that modifies the release or uptake of cysteamine into a subject following the administration of the compound, such as a reducing agent or a pantetheinase inducing agent.

Cysteamine is a small, highly reactive thiol molecule (NH2-CH2-CH2-SH) present in all life forms from bacteria to people. The IUPAC name for cysteamine is 2-aminoethanethiol. Other common names include mercaptamine, beta-mercaptoethylamine, 2-mercaptoethylamine, decarboxycysteine and thioethanolamine. In humans cysteamine is produced by the enzyme pantetheinase, which cleaves pantetheine into cysteamine and pantothenic acid, also known as pantothenate or vitamin B5. Human pantetheinases are encoded by the Vanin 1 and Vanin 2 genes (abbreviated VNN1 and VNN2) and are widely expressed, including in the gastrointestinal tract. Thus dietary pantetheine, which is present in many foods, (e.g. in nuts and dairy products), is cleaved in the gastrointestinal lumen to generate cysteamine and pantothenic acid, which are then absorbed. In particular, cysteamine can be transported across the gastrointestinal epithelium by organic cation transporters (OCTs), a family of transporters that includes organic cation transporter 1 (OCT1), OCT2 and OCT3, which have been shown to transport cysteamine in enterocytes. Based on its ability to be converted into cysteamine in the gastrointestinal tract pantetheine is a cysteamine precursor. Cysteamine precursors represent a class of compounds which can have advantages over cysteamine salts with respect to (i) tolerability and side effects, (ii) pharmacokinetics and dosing intervals, (iii) manufacturing and (iv) product stability. More generally, administering a cysteamine precursor from which cysteamine can be generated in vivo at varying rates, and using formulation methods to deliver those precursors to selected sites in the gastrointestinal tract at selected times, can be useful in a treatment regimen by providing much better control of cysteamine pharmacokinetics, which up until the present has been a major hindrance to wide spread use of cysteamine and other thiols.

### Cysteamine Precursor Compounds

Pantetheine, and its catabolic products cysteamine and pantothenate, are intermediate compounds in coenzyme A biosynthesis in plants and animals. Several compounds in the coenzyme A biosynthetic pathway such as 4-phosphopantetheine, dephospho-coenzyme A and coenzyme A, can be catabolized to pantetheine, and then to cysteamine and pantothenate, in the human gastrointestinal tract. Thus 4-phosphopantetheine, dephospho-coenzyme A and coenzyme A, by virtue of being convertible to cysteamine in the gut, are cysteamine precursors. N-acetylcysteamine is also a cysteamine precursor, via deacetylation either in the gut or by cellular deaceylases (e.g. the deacetylases which convert N-acetylcysteine to cysteine in vivo).

Pantethine is a dimer of two pantetheine molecules, joined by a disulfide bond (e.g., an oxidized form of pantetheine). The interconversion of pantethine into two pantetheines is not enzymatically mediated and does not require ATP. The reaction is instead controlled largely by the redox environment in the gut. In a reducing environment, which tends to prevail in vivo, particularly intracellularly, pantetheine will predominate, while in a more oxidizing environment, such as the stomach, the equilibrium will shift towards pantethine. A small clinical study by Wittwer (Wittwer et al., J. Exp. Med. 76:4 (1985)) showed that, when administered orally, a significant fraction of pantethine is chemically reduced to pantetheine in the human gastrointestinal tract, and subsequently cleaved to cysteamine and pantothenate. Thus, pantethine is a cysteamine precursor. Pantetheine herein refers to the D-enantiomer.

The pantothenoyl moiety of pantetheine contains a chiral carbon. Thus, there are two enantiomeric forms of pantetheine, traditionally referred to as D-pantetheine and L-panthetheine (also referred to as R-pantetheine and S-panthetheine). Only the D-enantiomer of pantetheine can be cleaved by pantetheinase, thus only the D-enantiomer qualifies as a cysteamine precursor. The two enantiomers of pantetheine can combine in four ways to form the disulfide pantethine: D-,D-; D-,L-; L-,D-; and L-,L-pantethine. Only D-,D-pantethine can be chemically reduced to two D-pantetheines and then cleaved to produce two cysteamines. Thus the D-,D-form of pantethine is strongly preferred, and the term pantethine as used herein refers to the D-,D-enantiomer. The pantetheine-related compounds 4-phosphopantetheine, dephospho-coenzyme A and coenzyme A also must be in the D-stereoisomeric configuration to yield D-pantetheine (and thence cysteamine) upon degradation in the gut. Therefore “4-phosphopantetheine”, “dephospho-coenzyme A” and “coenzyme A,” as well as any analogs or derivatives thereof, herein refer to the D-enantiomer. None of pantetheine, 4-phosphopantetheine, dephospho-coenzyme A or coenzyme A is absorbed by enterocytes, rather each compound must be catabolized to pantothenate and cysteamine which are absorbed (see Shibata et al., J. Nutr. 113:2107 (1983)).

Analogs or derivatives of the D-stereoisomer of pantetheine, 4-phosphopantetheine, dephospho-coenzyme A or coenzyme A that can be converted to the parent compound in the gastrointestinal tract (e.g. by natural enzymatic or chemical processes) can also be used to form either thiol or disulfide-type cysteamine precursors and are herein referred to as “suitable analogs or derivatives.” For example, there are many physiologic forms of coenzyme A (e.g. acetyl CoA, succinyl coA, malonyl coA, etc.) that are readily degraded to coenzyme A in the gut. Any acetylated, alkylated, phosphorylated, lipidated or other analog may be used as a cysteamine precursor. Analogs of pantetheine, 4-phosphopantetheine, dephospho-coenzyme A or coenzyme A have been described in the literature, as well as methods for producing them (van Wyk et al., Chem Commun 4:398 (2007)).

Pantetheine can form disulfides with thiols other than itself, referred to as pantetheine mixed disulfides, which constitute another class of cysteamine precursors. The thiols reacted with pantetheine are preferably naturally occurring thiols, or non-natural thiols known to be safe in man based on a history of human or animal use. For example, mixed disulfides can be formed by reacting pantetheine with 4-phosphopantetheine, dephospho-coenzyme A or coenzyme A, compounds present in the human body and in many foods. Such mixed disulfides, upon reduction and degradation in the gut yield two cysteamines. Pantetheine coupled to N-acetylcysteamine also yields two cysteamines upon reduction and degradation in the gut. In certain embodiments disulfide cysteamine precursors that can yield two cysteamines are preferred. Analogs or derivatives of 4-phosphopantetheine, dephospho-coenzyme A or coenzyme A that can be converted to the parent compound in the gastrointestinal tract via chemical or enzymatic processes (i.e. suitable analogs or derivatives) can also be coupled to pantetheine to form pantetheine mixed disulfide cysteamine precursors, or they can be coupled to other thiols.

Pantetheine mixed disulfides can also be formed by reacting pantetheine with thiols not themselves degradable to cysteamine, such as L-cysteine, homocysteine, N-acetylcysteine, N-acetylcysteine amide, N-acetylcysteine ethyl ester, N-acetylcysteamine, L-cysteine ethyl ester hydrochoride, L-cysteine methyl ester hydrochoride, thiocysteine, allyl mercaptan, furfuryl mercaptan, benzyl mercaptan, thioterpineol, 3-mercaptopyruvate, cysteinylglycine, gamma glutamylcysteine, gamma-glutamylcysteine ethyl ester, glutathione, glutathione monoethyl ester, glutathione diethyl ester, mercaptoethylgluconamide, thiosalicylic acid, thiocysteine, tiopronin or diethyldithiocarbamic acid.

Dithiol compounds such as dihydrolipoic acid (DHLA), meso-2,3-dimercaptosuccinic acid (DMSA), 2,3-dimercaptopropanesulfonic acid (DMPS), 2,3-dimercapto-1-propanol, bucillamine or N,N′-bis(2-mercaptoethyl)isophthalamide can also be reacted with pantetheine to form either a pantetheine mixed disulfide with one free thiol group, or a tripartite compound with two disulfide bonds connecting two pantetheine molecules to the dithiol. The former category of mixed pantetheine disulfides yields one cysteamine upon disulfide bond reduction and pantetheinase cleavage, while the latter category yields two cysteamines. Alternatively, two different thiols can be bonded to a dithiol to yield a cysteamine precursor, so long as one of the thiols is cysteamine, pantetheine, 4-phosphopantetheine, dephospho-coenzyme A, coenzyme A or N-acetylcysteamine, or a suitable analog or derivative thereof; that is, a compound which can ultimately be degraded to cysteamine in the gastrointestinal tract.

Similarly to pantetheine, any of 4-phosphopantetheine, dephospho-coenzyme A, coenzyme A or N-acetylcysteamine, or suitable analogs or derivatives, can be (i) reacted with itself to form a homodimeric disulfide, or (ii) reacted with each other in various pairs to form mixed disulfides, or (iii) reacted with other thiols (not convertible into cysteamine in vivo), to form mixed disulfides. All such disulfides are cysteamine precursors. The first two categories can yield two cysteamines upon reduction and degradation in the gut while the third category can yield only one cysteamine.

To summarize, cysteamine precursors can be classified in three main categories: (i) thiols degradable to cysteamine, (ii) mixed disulfides which include cysteamine, including disulfides formed with dithiols, (ii) disulfides which include pantetheine, (iii) disulfides which include 4-phosphopantetheine, dephospho-coenzyme A or coenzyme A or suitable analogs or derivatives. Each of the latter three categories can be further decomposed depending on the second thiol: (a) pantetheine or suitable analogs or derivatives, (b) 4-phosphopantetheine, dephospho-coenzyme A, or coenzyme A or suitable analogs or derivatives, or (c) a thiol which is not itself a cysteamine precursor (e.g. L-cysteine, homocysteine, N-acetyl-cysteine, N-acetylcysteine amide, N-acetylcysteine ethyl ester, N-acetylcysteamine, L-cysteine ethyl ester hydrochoride, L-cysteine methyl ester hydrochoride, thiocysteine, allyl mercaptan, furfuryl mercaptan, benzyl mercaptan, 3-mercaptopyruvate, thioterpineol, glutathione, cysteinylglycine, gamma glutamylcysteine, gamma-glutamylcysteine ethyl ester, glutathione monoethyl ester, glutathione diethyl ester, mercaptoethylgluconamide, thiosalicylic acid, thiocysteine, tiopronin or diethyldithiocarbamic acid). Dithiol compounds such as dihydrolipoic acid, meso-2,3-dimercaptosuccinic acid (DMSA), 2,3-dimercaptopropanesulfonic acid (DMPS), 2,3-dimercapto-1-propanol, bucillamine or N,N′-bis(2-mercaptoethyl)isophthalamide can also be combined with cysteamine, pantetheine, 4-phosphopantetheine, dephospho-coenzyme A or coenzyme A or suitable analogs or derivatives to form disulfides.

Pharmacological Properties of Cysteamine Precursors

The temporal and spatial pattern of in vivo cysteamine generation from cysteamine precursors can vary widely depending on the type of cysteamine precursor. Cysteamine precursors that require multiple chemical and enzymatic reactions to generate cysteamine will, on average, generate cysteamine later than those that require only one step. This property of cysteamine precursors can be used to design a plurality of pharmaceutical compositions with varying rates and durations of in vivo cysteamine creation. Further, the pharmaceutical compositions can be administered in combinations and in ratios that bring about desirable pharmacological ends. For example, to provide elevated plasma cysteamine levels shortly after drug administration a cysteamine mixed disulfide may be administered. The only step required to produce a cysteamine from a cysteamine mixed disulfide is reduction of the disulfide bond. Depending on the identity of the second thiol a second cysteamine may be produced, following one or more degradative steps. The second cysteamine can only be generated after disulfide bond reduction and another step, so it will necessarily be produced later than the first cysteamine, thereby extending the period of time over which cysteamine is generated in the gut and absorbed into the blood. Since cysteamine free base and cysteamine salts (e.g. Cystagon® and Procysbi®) have a very short half life this prolongation of cysteamine creation in vivo from cysteamine precursors represents a significant advance over present therapeutics.

In one approach, if the second thiol is pantetheine (i.e. a cysteamine-pantetheine disulfide) then a pantetheinase cleavage step is necessary to generate a second cysteamine. Pantetheinase is generally located on the surface of enterocytes, and thus is only in contact with a fraction of gut contents at any one time, thereby extending the period of time during which cysteamine is generated. This combination of early and late cysteamine generation from one disulfide molecule has several advantages: (i) cysteamine becomes available upon disulfide bond reduction, providing early therapeutic benefit, (ii) the cleavage of pantetheine occurs over time (pantetheinases are expressed at varying levels throughout the gastrointestinal tract), extending the duration of therapeutic benefit, (iii) the extended production of cysteamine over time and space, via both disulfide bond reduction and pantetheine cleavage, reduces the high peak cysteamine concentrations that are strongly associated with side effects, while also (iv) avoiding saturation of pantetheinase or cysteamine uptake mechanisms such as transport by OCTs. In short, the prolonged elevated blood cysteamine levels provide both a more efficacious medication and a less toxic and more convenient dosing form for patients.

Alternatively, if the second thiol is L-cysteine (i.e. a cysteamine-L-cysteine disulfide) then only one cysteamine is generated, upon reduction of the disulfide, and there is no long-duration cysteamine generation. However, as described below, the cysteamine-L-cysteine disulfide can be formulated for release in virtually any part of the gastrointestinal tract, including the ileum or colon, where a cysteamine precursor capable of rapid cysteamine release may be useful. Further, cysteine has also been shown to enhance the activity of pantetheinase, and to have beneficial effects in several disease models. Thus a cysteamine-L-cysteine disulfide may be a useful complement to another cysteamine precursor, or may be useful for treatment of diseases responsive to both cysteamine and cysteine.

Disulfides that contain a thiol requiring two or more catabolic reactions to generate cysteamine, such as 4-phosphopantetheine, dephospho-coenzyme A or coenzyme A, or suitable analogs or derivatives thereof, can be more efficiently degraded in the small intestine, where they are exposed to the digestive enzymes present in pancreatic juice, than in the stomach or large intestine. Disulfides made by reacting two such thiols with each other, or with thiols other than cysteamine, will generate cysteamine starting at a later time point and extending over a longer time period than, for example, a cysteamine-L-cysteine disulfide. On average 4-phosphopantetheine, dephospho-coenzyme A or coenzyme A, or suitable analogs will generate cysteamine later than pantetheine, and the same is true of disulfides containing those compounds.

Cysteamine precursors such as panthetheine and compounds degradable to pantetheine in the gut, as well as disulfides containing any of those compounds all yield pantothenate, along with cysteamine, upon cleavage by pantetheinase. Pantothenate, or vitamin B5, is a water soluble compound that is present in the diet and is synthesized by enteric bacteria. When pantothenate is administered in large doses the excess is excreted in urine. A review of panthothenate by the Panel on Folate, Other B Vitamins, and Choline of the US Institute of Medicine Standing Committee on the Scientific Evaluation of Dietary Reference Intakes (National Academies Press (US), 1998) found that: “No reports of adverse effects of oral pantothenic acid in humans or animals were found.”

Mixtures of Cysteamine Precursors

The methods of the invention can utilize mixtures of cysteamine precursors to take advantage of their differing pharmacological properties. In particular, individualized improvement (or personalization for a given patient's needs) of cysteamine plasma levels can be achieved by using mixtures of cysteamine precursors. For example, the cysteamine-pantetheine mixed disulfide described above fixes the ratio of cysteamine to pantetheine at 1:1. However cysteamine is absorbed and cleared from the body rapidly (elimination half life: ˜25 minutes), producing a sharp peak in blood levels, while pantetheine provides cysteamine (via pantetheinase cleavage) over several hours. Thus a dose of a cysteamine-pantetheine mixed disulfide that produces therapeutic cysteamine levels early (from the cysteamine released upon disulfide bond reduction) may produce sub-therapeutic cysteamine levels later, because cysteamine generation from pantetheine is spread over a longer period of time. Thus a 1:1 ratio of cysteamine:pantetheine may not be ideal for a specific patient or purpose. Adding more pantetheine to the dosage form would keep blood cysteamine in the therapeutic concentration range for a longer period of time. To increase the ratio of pantetheine to cysteamine, either the thiol pantetheine or the disulfide pantethine or another pantetheine-containing disulfide can, for example, be co-formulated or co-administered with the cysteamine-pantetheine mixed disulfide to achieve blood cysteamine levels in the therapeutic range for a longer period of time. The ratio of the two cysteamine precursors can be adjusted to achieve desired pharmacokinetic parameters, such as maximizing the area under the cysteamine concentration-time curve (AUC), or minimizing the peak concentration (Cmax) of cysteamine, or maximizing the trough concentration (Cmin), or maintaining cysteamine blood levels above a threshold, or any combination of such parameters.

Cysteamine precursors such as 4-phosphopantetheine, dephospho-coenzyme A or coenzyme A, and disulfides formed from those three compounds, require more catabolic steps to yield cysteamine than does pantetheine (which only requires one step). Accordingly, the rate of cysteamine production from those cysteamine precursors is, on average, slower and more prolonged than from pantetheine or certain pantetheine disulfides. Thus co-administration or co-formulation of 4-phosphopantetheine, dephospho-coenzyme A or coenzyme A, or their disulfides in combination with cysteamine-pantetheine, and optionally pantetheine or pantethine, provides another way to control cysteamine pharmacokinetics by selecting appropriate cysteamine precursors. In particular, use of such cysteamine precursors can be used to further extend the time over which cysteamine is produced in the gastrointestinal tract.

N-Acetylcysteamine Disulfide (Compound 3)

In certain embodiments the cysteamine precursor is compound 3 or a pharmaceutically acceptable salt thereof. The homodimer of two N-acetylcysteamines is an efficient delivery vehicle for cysteamine which can be used in two ways: it can be administered as a single agent, or in combination with one or more other cysteamine precursors. In both cases the goal is to provide sustained blood N-acetylcysteamine and cysteamine levels in the therapeutic range (e.g. greater than 5 micromolar but less than 75 micromolar, or greater than 10 micromolar but less than 65 micromolar in blood plasma) for the longest possible time.

In those embodiments in which compound 3 is administered as a single agent it is preferably formulated in a way that provides at least two release profiles: an early release profile and a later release profile. The early release formulation (aka instant release) starts to release compound 3 within ten minutes after oral administration. The later release formulation starts to release substantial amounts of compound 3 between about two to four hours later. The two formulations are admixed so they can be ingested together in a single dosage form. The ratio of the dose of compound 3 formulated for early release to that formulated for later release is at least 1:2, and may range up to 1:8 (e.g. 1:2, 1:3, 1:4, 1:5, 1:6, 1:7, 1:8). In one embodiment the early and late release dose components are both formulated as microbeads. Microbeads with the two release profiles can be manufactured separately then mixed together in the desired ratio to produce the dosage form (e.g., in a sachet). That approach facilitates manufacturing doses with different ratios of early to late release microbeads. Different ratios of the two types of microbeads can be used to individualize therapy for patients.

In some embodiments compound 3 is formulated in three release profiles: early, intermediate and late. The early release component starts to release compound 3 within ten minutes after oral administration, the intermediate release component starts to release substantial amounts of Compound 3 between about two to four hours after dose ingestion, and the late formulation about 3 to 6 hours after ingestion. The three release components are admixed so they can be ingested together in a single dosage form. The ratio of compound 3 in the three dose components (early:intermediate:late) is at least 1:2:2. The compound 3 in the intermediate and late components may vary independently between 2-8 times the amount in the early component, however the late release component is at least equal to the intermediate release component (e.g. 1:2:8, 1:4:6, 1:4:4, 1:5:5, 1:6:8 and so forth). In one embodiment the early, intermediate and late release dose components are all formulated as microbeads, which can be manufactured separately then mixed together in the desired ratio (e.g. a ratio customized to the gastrointestinal and liver physiology of an to produce the dosage form (e.g. in a sachet).

In certain embodiments the late dose component, or both the intermediate and late dose components are formulated for prolonged retention in the stomach (gastroretentive formulation). In other embodiments the late, or both the intermediate and late dose components are formulated for sustained release. In certain embodiments the two- or three-component dosage form is ingested with a meal, preferably a meal containing at least 500 calories, more preferably at least 700 calories. Preferably the meal is nutritionally complex (e.g. contains whole foods of several types) and at least 25% of the caloric content is derived from fat.

In those embodiments in which compound 3 is co-administered with at least one additional cysteamine precursor it is formulated to provide a release profile that complements the release profile of the at least one other cysteamine precursor, so that together the cysteamine precursors provide plasma cysteamine concentrations in the therapeutic range for the greatest length of time possible. In preferred embodiments compound 3 provides cysteamine in the first 1 to 3 hours after dosing and the at least one additional cysteamine precursor provides cysteamine for hours 3-6, 3-8, 4-10 or 3-12 of, for example, a 12 hour interval between doses. In such an embodiment the compound 3 may be formulated for instant release. In certain embodiments the at least one additional cysteamine precursor co-administered with compound 3 is compound 1 or a pharmaceutically acceptable salt thereof.

Testing of Cysteamine Precursors

Cysteamine precursors can be tested for efficacy using receptor binding and cell infection assays (see, e.g., Khanna et al, bioRxiv (preprint Dec. 8, 2020). Cysteamine precursors can decrease binding of SARS-CoV-2 spike protein to its receptor, decrease the entry efficiency of SARS-CoV-2 spike pseudotyped virus, and inhibit SARS-CoV-2 live virus infection (see, e.g., Suhail et al, Protein J. 39:644 (2020).

Enhancers of Cysteamine Production from Cysteamine Precursors

The methods of the invention can utilize enhancers of cysteamine production. Additional flexibility in controlling cysteamine blood levels can be achieved by combining cysteamine precursors with enhancers of the steps required to chemically and enzymatically break down cysteamine precursors to cysteamine in the gut, to absorb cysteamine into blood, and to prevent cysteamine from being rapidly catabolized in the gut, the blood or in tissues. Specific enhancers exist for each of these several steps. Thus any of the cysteamine precursors described herein may optionally be co-formulated or co-administered or administered in sequence with an agent that enhances cysteamine generation or intestinal uptake or slows cysteamine breakdown.

The first step in converting disulfide cysteamine precursors to cysteamine is reduction of the disulfide to produce two thiols. The redox environment in the gastrointestinal tract may not contain sufficient reducing equivalents to quantitatively reduce cysteamine precursors to their respective thiols, thereby limiting cysteamine generation. For example, the concentration of the reducing agents glutathione and cysteine in gastric juice is very low or undetectable (see Nalini et al., Biol Int. 32:449 (1994)). Further, in a small clinical study of high dose pantethine much of the pantethine was excreted unchanged in the stool, apparently reflecting incomplete disulfide bond reduction (see Wittwer et al., J. Exp. Med. 76:4 (1985)). To address this potential constraint, reducing agents may be co-administered or co-formulated with disulfide cysteamine precursors, or administered before or after cysteamine precursors so they are available at the time and in the place where needed. Reducing agents may promote disulfide bond reduction, freeing two thiols, or they may promote thiol-disulfide exchange reactions, in which a thiol (A) and a disulfide (B-C) react to produce a new disulfide (A-B or A-C) and a thiol (B or C), thereby releasing one of the thiols in the original disulfide (e.g. cysteamine, pantetheine or a compound degradable to cysteamine).

A variety of reducing agents may be used to promote reduction of disulfides, or thiol-disulfide exchange, in the gastrointestinal tract. Reducing agents may either directly reduce disulfide cysteamine precursors or they may reduce other disulfides, such as glutathione disulfide, that in turn reduce disulfide cysteamine precursors or participate in thiol-disulfide exchanges. In some embodiments physiological compounds (i.e. substances normally found in the body) or food-derived compounds with reducing capacity may be used to promote reduction of disulfide cysteamine precursors, or to promote thiol-disulfide exchange reactions. Physiologic reducing agents such as the thiols glutathione or cysteine (both present in the small intestine as a result of bile and enterocyte secretion) may be used, as may other compounds normally present in the body and in food such as ascorbic acid (vitamin C), tocopherols (vitamin E) or the dithiol dihydrolipoic acid, a potent reducing agent. Other widely available reducing agents including thiols such as N-acetylcysteine and non-thiols such as nicotinamide adenine dinucleotide (NADH), may also be used. Preferred reducing agents include those known to be safe in the doses required to bring about a change in the local gastrointestinal redox environment. Up to several grams of reducing agent may be required per dosing period, for example 0.5-5 grams. In particular, the compounds 1-3 can benefit from the co-administration or appropriately times subsequent administration of one or more reducings agents, as described herein. Two or more reducing agents may be combined. Preferably reducing agents have a molecular mass less than 300 Daltons.

Adult humans produce between 400 to over 1,000 milliliters (ml) of bile daily; 750 ml has been estimated as an average volume (Boyer, Compr. Physiol. 3:32 (2013)). Bile is produced in the liver throughout the day. Some is stored in the gall bladder, while the remainder provides a steady slow flow of bile, even in the fasted state (bile serves an excretory function as well as aiding in digestion and fat absorption). A meal stimulates duodenal secretion of the peptide hormones secretin and cholecystokinin, and they stimulate bile production and gall bladder contraction, respectively. The concentration of thiols in bile is approximately 4 mM, consisting mostly glutathione but also including gamma-glutamylcysteine, cysteinylglycine and cysteine (Eberle et al., J Biol. Chem. 256:2115 (1981); Abbott & Meister, J. Biol. Chem 258:6193 (1984))

Cysteine and, to a lesser extent, glutathione are also secreted into the lumen of the gastrointestinal tract by enterocytes to regulate the luminal redox potential. The thiol concentration in intestinal fluid from the jejunum of rats has been measured directly, independent of contributions from bile. It ranges from 60-200 μM in fasted rats and from 120-300 μM in fed animals (Hagen et al., Am. J. Physiol. 259:G524 (1990); Dahm and Jones, Am. J. Physiol. 267:G292 (1994)). Furthermore, unlike bile secretion, the maintenance of luminal thiol levels is a dynamic process, so that increases in intestinal levels of oxidized molecules (such as disulfide cysteamine precursors) may be countered, at least to some extent, by increased cysteine production by enterocytes (Dahm and Jones, J. Nutr. 130:2739 (2000)). The human small intestine secretes about 1.8 liters of fluid per day, and the colon about 0.2 liters, for a total of about 2 liters. The concentration of thiols (mainly cysteine) in the secreted fluid varies according to the region of the gastrointestinal tract, luminal redox potential and diet.

The total concentration of gastrointestinal thiols (both bile and enterocyte-derived) will affect the rate and extent of disulfide bond reduction and/or thiol-disulfide exchange necessary to convert cysteamine precursors to thiols, which is the necessary first step in their degradation to cysteamine. The amount of reducing equivalents available in the upper gastrointestinal tract following a meal can be estimated by making a few assumptions. For example, if we assume (i) 200 ml of bile is secreted in the hour following a large meal, and a further 100 ml in the following 2-3 hours, and (ii) the thiol concentration in bile is 4 mM, then the milliequivalents of thiol reducing power in bile amount to 0.3 L×0.004 moles/L=0.0012 moles of thiol (1.2 millimoles). Further assume that small intestinal enterocytes secrete an additional 400 milliliters during the four hours following a meal, with a thiol concentration of 200 uM, providing an additional 0.4 liters×0.0002 moles/liter=80 micromoles of luminal thiols. Combined with bile thiols a total of ˜1.28 millimoles are available to reduce dietary disulfides and maintain intestinal redox potential. This is not an estimate of the upper limit of thiol secretion, which may be considerably greater, but of the normal levels of thiols in the small intestine in the hours after a meal.

A 0.5 gram dose of cysteamine-(R)-pantetheine disulfide (MW: 353.52 g/L) contains ˜1.41 millimoles of disulfide bonds, and could therefore, in principal, be converted to thiols (either via disulfide bond reduction or thiol-disulfide exchange) by endogenous levels of thiols (ignoring the need for luminal thiols for other physiological purposes).

More generally, cysteamine precursor doses in excess of 1.25 millimoles may benefit from co-administration of an exogenous reducing agent. Many natural products, normally present in the diet, can provide reducing power to facilitate cysteamine precursor reduction or thiol-disulfide exchange, including the principal endogenous intestinal thiols cysteine or glutathione. Cysteine or glutathione analogs may also be used, such as N-acetylcysteine, N-acetylcysteine ethyl ester or N-acetylcysteine amide. Ascorbic acid is another agent that can reduce disulfide bonds (Giustarini et al. Nitric Oxide 19:252 (2008)). The dose of ascorbic acid required to provide reducing power equivalent to, for example, 1 gram of the disulfide cysteamine precursor cysteamine-(R)-pantetheine disulfide can be calculated as follows:

The molecular weight of ascorbic acid (176.12 g/mol) is roughly half that of cysteamine-(R)-pantetheine disulfide, also known as compound 1 (353.52 g/mol). Thus 1 gram of ascorbic acid has equimolar reducing equivalents to the number of disulfide bonds in a 2 gram dose of compound 1. Although the daily intake of vitamin C recommended by the U.S. Food and Nutrition Board is only 75 milligrams for women and 90 milligrams for men, many people take much higher doses, including doses of 1 gram per day or more, with apparently few or no adverse effects.

Similar reasoning provides the amounts of other reducing agents needed to match a compound 1 dose in molar terms. For example cysteine (molecular weight: 121.15 Daltons) is about 34% of the mass of compound 1; N-acetylcysteine (molecular weight: 163.195 Daltons) is about 46% of the mass of compound 1; alpha lipoic acid (molecular weight: 208.34 Daltons) is about 59% of the mass of compound 1, and so forth. Alpha lipoic acid and N-acetylcysteine are widely available in vitamin stores and on the internet in 600 and 1,000 mg capsules and tablets, respectively, including sustained release formulations, indicating their non-regulated status. Similar calculations can be made for other disulfide cysteamine precursors based on their molecular weight.

Because bile is the main source of thiols, and bile is successively diluted along the length of the small and large intestines, extra reducing power for cysteamine precursor reduction may be more useful in the jejunum, ileum or colon than in the duodenum. Hence formulations designed to release reducing agents in the distal small intestine and/or large intestine may be particularly useful supplements to disulfide cysteamine precursors. Sustained release formulations of ascorbic acid and other reducing agents are commercially available. Alternatively ascorbic acid could be co-formulated with a cysteamine precursor to ensure co-delivery of both agents.

The electrochemical potentials (reducing strength) associated with different biological reducing agents are known, and provide a guide to their use, however the capacity of such agents to reduce different disulfide cysteamine precursors is best determined empirically.

The kinetics of thiol-disulfide exchange reactions are strongly influenced by pH (i.e. retarded by low pH). Such exchange reactions are an alternative mechanism to disulfide bond reduction for freeing cysteamine from a cysteamine mixed disulfide, or pantetheine from a pantetheine disulfide, and so forth. To enhance the kinetics of thiol-disulfide exchange reactions basic compounds may be co-administered or co-formulated with disulfide cysteamine precursors, so they are available at the time and place where needed. Physiological compounds such as bicarbonate, present at high concentrations in pancreatic juice, may be used to modulate local gastrointestinal pH.

An essential step in converting many cysteamine precursors to cysteamine is the enzyme pantetheinase, encoded by the VNN1 and VNN2 genes in man. Pantetheine and pantetheine disulfides, including pantethine, require this enzyme to yield cysteamine. Pantetheinase is also ultimately required for cysteamine generation from compounds convertible into pantetheine in the gastrointestinal tract, such as 4-phosphopantetheine, dephospho-coenzyme A, coenzyme A and suitable analogs and derivatives. Normal levels of pantetheinase in the gastrointestinal tract may not be adequate to quantitatively cleave all the pantetheine molecules provided by pharmacological doses. To address this constraint, compounds that induce pantetheinase expression can be co-administered or co-formulated with cysteamine precursors that contain pantetheine, or compounds convertible into pantetheine, to increase the amount of pantetheinase in the gastrointestinal tract at the time and place where needed (i.e. when and where pantetheine is present). Agents that induce expression of pantetheinases include both physiological substances, including certain food components, and pharmacological agents, including FDA approved drugs. Physiological inducers of VNN1 include a variety of substances that act via the transcription factors NF-E2-related factor-2 (more commonly referred to by the acronym Nrf2), peroxisome proliferator activated receptor alpha (PPAR alpha) and peroxisome proliferator activated receptor gamma (PPAR gamma).

Factors that induce Nrf2 activation (via translocation to the nucleus) include both natural products and certain drugs. For example, sulforaphane, an isothiocyanate present in cruciferous vegetables, such as broccoli, Brussels sprouts, cabbage and cauliflower, induces VNN1 expression via Nrf2. Foods rich in sulforaphane (e.g. broccoli sprouts) may be used to induce pantetheinase expression, or sulforaphane can be administered as a pure substance in a pharmaceutical composition. Certain food-derived thiols, including S-allyl cysteine and diallyl trisulfide (both present in onions, garlic and garlic extract) also induce Nfr2, and can be included in meals administered with cysteamine precursors. Alternatively either compound may be obtained in pure form and administered in a pharmaceutical composition. Lipids present in certain foods, including some polyunsaturated fatty acids, oxidized fat, omega-3 fatty acids and the naturally occurring lipid oleoylethanolamide (OEA) also induce Nrf2 and/or PPAR alpha. Foods rich in oxidized fat include French fries and other deep fried foods, which can be coadministered with cysteamine precursors that require pantetheinase cleavage to generate cysteamine. Omega-3 fatty acids are present in fish and available in fish oil extracts and in pure form for use in pharmaceutical compositions.

Naturally occurring PPAR alpha ligands include endogenous compounds such as arachidonic acid and arachidonic acid metabolites including leukotriene B4, 8-hydroxyeicosatetraenoic acid and certain members of the family. Pharmacological PPAR alpha ligands include the fibrates (e.g. benzafibrate, ciprofibrate, clinofibrate, clofibrate, fenofibrate, gemfibrozil), pirinixic acid (Wy14643) and di(2-ethylhexyl) phthalate (DEHP). Any natural or synthetic PPAR alpha ligand may be co-formulated or co-administered with a cysteamine precursor which requires pantetheinase cleavage to produce cysteamine. For a review of PPAR ligands see Grygiel-Gorniak, B. Nutrition Journal 13:17 (2014).

Natural and synthetic PPARG agonists may also be used to stimulate Nrf2-mediated transcription of the pantetheinase genes VNN1 and/or VNN2. Natural product PPARG agonists include arachidonic adid and metabolites including 15-hydroxyeicosatetraenoic acid (15(S)-HETE, 15(R)-HETE, and 15(S)-HpETE), 9-hydroxyoctadecadienoic acid, 13-hydroxyoctadecadienoic acid, 15-deoxy-(delta)12,14-prostaglandin J2 and prostaglandin PGJ2, as well as honokiol, amorfrutin 1, amorfrutin B and amorphastilbol. Other natural products activate both PPARG and PPARA, including genistein, biochanin A, sargaquinoic acid, sargahydroquinoic acid, resveratrol and amorphastilbol. Natural product PPARG agonists are described and reviewed in Wang et al., Biochemical Pharmacology 92:73 (2014)). Pharmacological PPAR gamma agonists include thiazolidinediones (also called glitazones, e.g. pioglitazone, rosiglitazone, lobeglitazone). Heme, derived from red meat, also induces VNN1 expression. PPARA or PPARG agonists that stimulate pantetheinase expression may be co-administered or co-formulated with cysteamine precursors containing pantetheine or a compound degradable to pantetheine in the gut. Two or more inducers of pantetheinase expression may be combined to enhance expression or to reduce the dose of any single agent.

Another important step in making cysteamine bioavailable throughout the body is absorption across the intestinal epithelium. Cysteamine uptake from the intestinal lumen is mediated by transporters, natural levels of which may not be sufficiently high to transport all cysteamine in the intestinal lumen. Accordingly, compounds that induce expression of cysteamine transporters can be co-administered or co-formulated with cysteamine precursors to enhance cysteamine absorption. Cysteamine is transported across the intestinal epithelium by organic cation transporters 1, 2 and 3 (encoded by the OCT1, OCT2 and OCT3 genes, also referred to as the SLC22A1, SLC22A2 and SLC22A3 genes) and possibly by other transporter proteins. Inducers of organic cation transporter expression include the transcription factors PPAR alpha and PPAR gamma, the pregnane X receptor (PXR), retinoic acid receptor (RAR) and (in the case of OCT1) the RXR receptor, as well as by the glucocorticoid receptor. Accordingly, either natural or synthetic ligands of these receptors can be used to increase OCT expression and consequently enhance cysteamine uptake by intestinal epithelial cells. Agents that stimulate expression of cysteamine transporter(s) may be co-administered or co-formulated with cysteamine precursors of any type.

The elimination half life of cysteamine in the human body (time from Cmax to half Cmax after an intravenous bolus) is about 25 minutes. Some of the cysteamine dose is transformed into a variety of disulfides, including mixed disulfides with free cysteine, with cysteinyl residues of proteins and with glutathione. No pharmacological intervention can prevent that mode of elimination, and in any event that pool of cysteamine remains available for further disulfide exchanges. There is a cysteamine catabolic pathway, however, that irreversibly transforms cysteamine, effectively removing it from the body. The enzyme cysteamine dioxygenase, which oxidizes cysteamine to hypotaurine, is a significant factor in cysteamine elimination. Hypotaurine is subsequently further oxidized to taurine. Co-administration of a cysteamine precursor with one or both of these catabolic products may slow cysteamine catabolism by end-product inhibition. Thus in certain embodiments a cysteamine precursor is co-formulated, co-administered or administered in optimal temporal sequence with hypotaurine and/or with taurine.

In summary, flexibility in controlling cysteamine blood levels can be achieved by co-formulation or co-administration of (i) one or more cysteamine precursors with selected properties, (ii) one or more enhancers of in vivo cysteamine precursor breakdown and/or cysteamine absorption (iii) one or more inhibitors of cysteamine catabolism, using (iv) one or more types of formulation (e.g. immediate, delayed, sustained, gastroretentive or colon-targeted or a combination) and (v) a dosing schedule that enables optimal co-delivery of cysteamine precursor(s) and enhancer(s) to targeted segments of the gastrointestinal tract in amounts that can be effectively degraded and absorbed. The consequence of individualized application of these tools is sustained cysteamine blood levels in the therapeutic range for a prolonged period, resulting in a superior pharmacological effect on disease compared to existing compounds and formulations.

### Pharmaceutical Compositions

The methods of the invention can utilize pharmaceutical compositions formulated to achieve a therapeutically effective plasma concentration of cysteamine over an extended period of time in order to: (i) reduce the side effects associated with high peak concentrations of cysteamine, (ii) reduce undertreatment caused by sub-therapeutic trough concentrations of cysteamine and (iii) improve patient convenience and hence compliance with therapy by reducing the number of doses per day. The compounds and formulations of the invention are also designed to (i) provide improved organoleptic properties compared to existing cysteamine formulations, (ii) reduce contact of free cysteamine with the gastric epithelium, a known source of gastrointestinal side effects, (ii) minimize the dose of cysteamine precursor required to achieve therapeutic cysteamine blood levels by matching the dose and delivery site(s) with the relevant digestive and absorptive processes in the gastrointestinal tract, which purpose may be achieved by (iii) optimizing cysteamine precursor breakdown and absorption by co-formulation or co-administration with enhancers of those processes.

For the compositions of the invention, a pharmaceutical excipient is included in all formulations to prevent exposure of a cysteamine precursor, or a salt thereof, in the mouth. Formulation methods for masking bitter or other unpleasant tastes include coatings, which may be applied in several layers. Flavorants and dyes may also be used. Methods for producing pharmaceutical compositions with acceptable mouth feel and/or taste are known in the art (e.g. see textbooks on pharmaceutical formulation, cited elsewhere; the patent literature also provides methods for producing organoleptically acceptable pharmaceutical compositions (see, e.g., U.S. Patent Publication No. 20100062988).

Gastroretentive Compositions

A first composition provides a cysteamine precursor, or a salt thereof, in a gastroretentive formulation. A variety of gastroretentive technologies are known in the art, several of which have been successfully used in marketed products. For reviews see, e.g., Pahwa et al., Recent Patents in Drug Delivery and Formulation, 6:278 (2012); and Hou et al., Gastric retentive dosage forms: a review. Critical Reviews in Therapeutic Drug Carrier Systems 20:459 (2003).

A gastroretentive formulation provides sustained release of a cysteamine precursor in the stomach. Depending on the type of cysteamine precursor subsequent in vivo cysteamine generation may start in the stomach, or in the small intestine, which is the tissue from which cysteamine is most efficiently absorbed. Some cysteamine precursors may continue to be converted into cysteamine in the large intestine, even if release from a pharmaceutical composition in the stomach or small intestine. For example, disulfide cysteamine precursors released in the stomach may remain predominately in the oxidized state in the acidic, oxidizing environment of the stomach, then start to release cysteamine after encountering reducing agents (e.g. biliary glutathione) in the small intestine. The gastroretentive composition will yield elevated blood cysteamine levels during hours 1-4 after ingestion, preferably hours 1-6, more preferably hours 1-8, hours 1-10, or longer.

Contrary to what is recommended for cysteamine bitartrate (see, for example, Procysbi® FDA Full Prescribing Information) gastroretentive formulations of cysteamine precursors should be administered with food, preferably with a meal containing sufficient caloric content and nutrient density to slow gastric emptying. A nutrient dense meal triggers osmoreceptors and chemoreceptors in the small intestine (and to a lesser extent in the stomach) which has the effect of stimulating neural and hormonal signals which diminish gastric motility, thereby delaying emptying. Delaying gastric emptying is a mechanism for prolonging the effect of a gastroretentive composition. However, filling the stomach with a large volume of food or liquid tends to promote gastric motility and speed up emptying, thus nutrient density is a more important property of a meal than volume. Solid food, which must be ground into small particles in the antrum and pylorus before emptying into the duodenum, prolongs gastric residence compared to liquid or semi-liquid food. Among liquid foods high viscosity liquids may slow gastric emptying relative to low viscosity liquids. Food with high osmotic content triggers duodenal osmoreceptors to transmit signals that slow gastric emptying. The release of cysteamine precursors in the stomach (e.g. from a gastroretentive formulation) may increase the osmolarity of the gastric contents, and hence the duodenal contents.

In certain embodiments disulfide cysteamine precursors are preferred for gastroretentive formulations because the acidic, oxidizing environment of the stomach tends to maintain disulfides in their oxidized form, thereby limiting exposure of the gastric epithelium to cysteamine, which is believed to be one cause of cysteamine toxicity. Upon entering the duodenum and mixing with bile, which contains a high (millimolar) concentration of glutathione, cysteine and other reducing agents, the disulfide will be reduced, thereby producing free thiols in a location where they are exposed to pantetheinases and where cysteamine transporters are expressed on enterocytes.

The presence of fat in the small intestine is the most potent known inhibitor of gastric emptying, and leads to relaxation of the proximal stomach and diminished contractions in the pyloric region. Once the fat has been absorbed in the small intestine and is no longer triggering inhibitory signals to the stomach, gastric motility resumes its normal pattern. Gastroretentive formulations may therefore ideally be administered with meals containing fatty foods. Protein-rich meals also slow gastric emptying but to a lesser extent, and carbohydrate rich meals still less.

Gastroretentive compositions may also be administered with compounds that slow gastric emptying, including certain lipids, for example fatty acids with at least 12 carbon atoms stimulate cholecystokinin release from enteroendocrine cells, reducing gastric motility, while fatty acids with shorter carbon cells are not as effective. In some embodiments food or a meal may be supplemented with fatty acids or triglycerides containing fatty acids with carbon chains of 12 or longer (e.g. oleic acid, myristic acid, triethanolamine myristate, a fatty acid salt).

Fat and protein, when they reach the duodenum, stimulate secretion of several gut hormones, including ghrelin, cholecystokinin (CCK) and glucagon-like peptide 1 (GLP1). CCK slows gastric emptying by binding the CCK1 receptor (abbreviated CCK1R, formerly called the CCK-A receptor). In some embodiments orally active CCK agonists or mimics, positive allosteric modulators of CCK1R, or agents that promote release of endogenous CCK, or that inhibit CCK degradation, or that otherwise prolong CCK action through some combination of those or other mechanisms, are administered with gastroretentive compositions to slow gastric emptying and prolong gastric residence of the gastroretentive composition. CCK is a peptide that exists in several forms ranging from 8 amino acids up to 53 amino acids (e.g. CCK-8, CCK-53). Oral administration of the peptides is not effective because they are digested in the gastrointestinal tract. Small molecule CCK agonists have been developed and tested by several research groups. For example SR-146,131 and related compounds were developed by scientists at Sanofi (U.S. Pat. Nos. 5,731,340 and 6,380,230, herein incorporated by reference).

Certain protease inhibitors induce CCK production or release, or prolong its half life, or otherwise potentiate its effect, including both food-derived mixtures and pure compounds. For example ingestion of a protease inhibitor concentrate derived from potato is associated with elevated levels of CCK, as is ingestion of soybean peptone and soybean beta-conglycinin peptone. Camostate is a synthetic protease inhibitor with pleiotropic effects, including stimulation of endogenous CCK release, and consequent slowing of gastric emptying. Camostat mesilate is a pharmaceutical salt that has been used extensively in man. FOY-251 is an active metabolite of camostat. In some embodiments an agent that stimulates CCK production or release, or prolongs CCK half life, or otherwise potentiate CCK effect is co-formulated or co-administered with a gastroretentive composition in an amount that slows gastric emptying. In some embodiments, camostat, FOY-251, or a prodrug, derivative or active metabolite of camostat, or a pharmaceutically acceptable salt thereof, is co-formulated or co-administered with a gastroretentive composition in an amount ranging between 50-300 mg/kg, or between 100-250 mg/kg.

Gastric emptying is also slowed by acidification of the chyme. For example citric and acetic acids have been shown to delay gastric emptying. In some embodiments food or a meal includes a natural source of citric acid (e.g. fruit or juice from an orange, lemon, lime, grapefruit or other citrus rich fruit) or acetic acid (e.g. vinegar, pickles or other pickled vegetables) or lactic acid (e.g. sauerkraut or kimchi). In some embodiments an amount of acidic food or liquid sufficient to lower the pH of gastric chyme below pH 4 or below pH 3.5 is administered with a gastroretentive composition.

Glucagon-like peptide-1 (GLP1) is another gut hormone that is released by cells in the duodenum in response to food, particularly ingested fat, and that influences gastric emptying. Orally administered GLP1 receptor agonists have been discovered by several research groups (e.g. Sloop et al., Diabetes 59:3099 (2010)). Positive allosteric modulators of the GLP1 receptor, which are not agonists themselves but which potentiate endogenous GLP1, are another category of GLP1R stimulating agents (e.g. Wootten et al., J. Pharmacol. Exp. Ther. 336:540 (2011); Eng et al., Drug Metabolism and Disposition 41:1470 (2013); also see U.S. Patent Publication Nos. 20060287242, 20070021346, 20070099835, 20130225488 and 20130178420, each of which is incorporated herein by reference). Among the compounds that positively modulates GLP-1 receptor signaling in the presence of endogenous GLP1 is quercetin, which acts by binding an allosteric site on the GLP-1 receptor and positively influencing receptor signaling upon binding of endogenous ligands (GLP-1, a peptide, is present in several forms.) Some quercetin analogs are also positive modulators of endogenous GLP1. Quercetin is a flavonol present in many fruits, vegetables, leaves and grains. It is used as an ingredient in health supplements, beverages and foods. In some embodiments a GLP-1 receptor agonist or positive allosteric modulator of GLP-1 is co-formulated or co-administered with a gastroretentive composition in an amount sufficient to delay gastric emptying. In some embodiments the GLP-1 receptor agonist or positive allosteric modulator is quercetin or an analog, derivative or active metabolite of quercetin. Certain small molecule drugs are also able to slow gastric emptying time, and may be co-administered or co-formulated with gastroretentive compositions.

Gastric emptying is also slowed by acidification of the chyme. For example citric and acetic acids have been shown to delay gastric emptying. In some embodiments, food or a meal includes a natural source of citric acid (e.g. orange, grapefruit or other citrus rich fruits) or acetic acid (e.g. vinegar, pickles or other pickled vegetables) or lactic acid (e.g. sauerkraut or kimchi). In some embodiments the pH of the chyme is reduced below 4 or below 3.5 by administration of acidic food or liquid with a gastroretentive composition.

U.S. Pat. No. 8,741,885 describes a method for prolonging gastric retention of a gastroretentive pharmaceutical composition (e.g. a floating, swelling or mucoadhesive composition) by combining an active pharmaceutical ingredient with an opioid. The purpose of the co-formulated opioid is to slow gastric emptying. Gastroparesis, or severely depressed gastrointestinal motility, is a well known and potentially serious complication of opioid therapy.

Sustained Release Compositions

A second composition provides a cysteamine precursor, or a salt thereof, in a non-gastroretentive sustained release formulation. Sustained release formulations are well known in the art: Wen, H. and Park, K. (editors) Oral Controlled Release Formulation Design and Drug Delivery: Theory to Practice. Wiley, 2010; Augsburger, and L. L. and Hoag, S. W. (editors) Pharmaceutical Dosage Forms—Tablets, volume 3: Manufacture and Process Control. CRC Press, 2008. The sustained release component may be a tablet, a powder, or a capsule filled with microparticles. Optionally the particles may vary in size, in composition (e.g the type or concentration of a sustained release polymer), or in the type or thickness of a coating agent, or in the number and composition of layers if coated with multiple layers of coating agents, such that drug is released at different rates, or at different starting times, from individual particles, thereby providing, in aggregate, drug release over an extended period of time compared to a formulation in which all particles are substantially identical. The sustained release formulation may optionally be coated with a pH sensitive material that prevents dissolution in the stomach (referred to as an enteric coating). The microparticles in a single composition may vary in the type or thickness of one or more coating agents. For example, the pH at which the coating dissolves may very. The two or microparticles used in such mixed compositions may be manufactured separately to tight specifications and then blended in a ratio to achieved prolonged drug release in vivo.

A sustained release composition may provide prolonged release of the cysteamine precursor in the stomach and/or the small intestine (not the former if enteric coated) and consequently sustained in vivo cysteamine generation. A sustained release formulation may be designed to release drug for a period of time roughly equal to the sum of the average gastric and small intestinal transit times, e.g. 3-5 hours if administered in the fasting state or 5-8 hours if administered with food or with a meal. Alternatively the sustained release formulation may be designed to release drug for longer than the sum of the average stomach and small intestinal transit times, so as to continue to release cysteamine precursors in the large intestine. In some embodiments such a sustained release composition may release a cysteamine precursor for between 4-8 hours when administered in the fasted state or between 6-10 hours, or longer, when administered with a meal.

The sustained release formulation may yield elevated blood cysteamine levels during hours 1-4 after ingestion, preferably hours 1-6, more preferably hours 1-8, still more preferably hours 1-10 or longer. Sustained release formulations of cysteamine precursors may be administered with food or between meals, and optionally with enhancers of cysteamine precursor degradation or cysteamine absorption. Food tends to inhibit absorption of free cysteamine, particularly fatty foods, and it is generally recommended to ingest cysteamine salts on an empty stomach, though small amounts of applesauce or similar foods are permitted.

Mixed Formulations

Some compositions necessarily have elements of two types of formulation, one mainly directed at controlling the rate of drug release and the other mainly directed at controlling the anatomical site of drug release. For example gastroretentive formulations always contain drug in a sustained release formulation; otherwise there would be no point in prolonged gastric residence. However, there are ways to combine immediate and sustained release components in a single gastroretentive formulation. For example, the immediate release component may form an outer layer that is rapidly dissolved or that rapidly disintegrates in the stomach, leaving a core sustained release component that remains in the stomach by one or more of the gastroretentive mechanisms described herein. However, not all types of formulation can be productively combined. For example an enteric coated gastroretentive formulation would be counterproductive because gastroretentive formulations are designed to release drug in the stomach—and gastric release would be blocked by a coating resistant to dissolution in acidic medium.

Compositions with different temporal or anatomical drug release profiles can, when combined with suitable cysteamine precursors, and optionally with enhancers of cysteamine generation or absorption, provide blood cysteamine levels in the therapeutic range for 0.5-6 hours, more preferably 0.5-8 hours, and most preferably 0.5-12, 0.5-15 hours or longer. Examples of productive combinations of formulations follow, including mixed formulations with up to two drug release components, and separately formulated compositions that can be combined in various amounts and ratios to tailor the amount and timing of in vivo cysteamine generation and absorption to the needs of an individual patient.

A third composition provides a mixed formulation of a first enteric coated component formulated for delayed release of a cysteamine precursor, or a salt thereof, in the small intestine; and a second component of enteric coated microparticles formulated for sustained release of a cysteamine precursor, or a salt thereof throughout the small intestine and the proximal part of the large intestine. The mixed formulation provides a first component to initially achieve elevated levels of cysteamine in the blood, while the second component sustains cysteamine levels in the blood over time.

A fourth composition provides a mixed formulation that includes (i) a sustained release gastroretentive formulation of a cysteamine precursor, or a salt thereof, (ii) an immediate release formulation of a cysteamine precursor, or a salt thereof designed to release drug in the stomach. The second component of the mixed formulation is on the exterior surface of the composition and starts to dissolve immediately on contact with the stomach contents. It is the first to generate cysteamine, albeit not necessarily in the stomach. The first (gastroretentive) component provides prolonged cysteamine precursor release in the stomach, and ensuing in vivo cysteamine generation throughout the small intestine and, depending on the characteristics of the cysteamine precursor, into the large intestine. The combined in vivo generation and absorption of cysteamine from the two components starts within 1 hour after administration of the mixed composition and continues for at least 5 hours, preferably remaining within the therapeutic concentration range for 8, 10, 12 or more hours.

In a fifth composition, a first component is formulated for immediate release in the stomach and includes a cysteamine precursor, preferably a cysteamine mixed disulfide or a pantetheine disulfide, or a salt thereof and a second component is formulated for sustained release of a cysteamine precursor, or a salt thereof. The first component is on the exterior surface of the composition, so that the second component remains intact after dissolution or disintegration of the first component. The mixed formulation of this fifth composition may produce an initial elevation of plasma cysteamine concentration from the immediated release component and maintain elevated levels of cysteamine from the second (sustained release) component, with continued in vivo cysteamine production for 6 hours, 8 hours, 10 hours of longer. The release of a cysteamine precursor (or several different cysteamine precursors) along the gastrointestinal tract, from the stomach to the large intestine allows the amount of cysteamine precursor to be matched to the levels of panthetheinase and cysteamine transporters in all segments of the gut, thereby maximizing cysteamine generation and absorption. Continuous intestinal generation and absorption of cysteamine avoids reliance on a high Cmax for lengthening exposure, thereby lessening cysteamine side-effects associated with high peak levels. Thus, mixed formulations of cysteamine precursors allow for administration of cysteamine to numerous disorders that are sensitive to the effects of cysteamine.

In a sixth composition, a first component is formulated for immediate release in the stomach and includes a cysteamine precursor, preferably a cysteamine mixed disulfide or a pantetheine disulfide, or a salt thereof; a second component is formulated for release of a cysteamine precursor, or a salt thereof in the ileum and/or colon. The mixed formulation of this sixth composition may produce an initial elevation of plasma cysteamine levels from the immediated release component and a second elevation of plasma cysteamine levels from the ilium and colon-targeted component around the time the first peak is rapidly decreasing. The second component may start to release cysteamine precursor four to eight hours after administration, depending on whether it was administered with or without food. The controlled release of a cysteamine precursor (or different cysteamine precursors) along the gastrointestinal tract, from the stomach to the large intestine allows the amount of cysteamine precursor to be matched to the levels of panthetheinase and cysteamine transporters in all segments of the gut to maximize cysteamine generation and absorption.

### Synthesis of Cysteamine Precursor Compounds

The pharmaceutically acceptable compositions of the invention include one or more cysteamine precursors, or pharmaceutically acceptable salt(s) thereof. Salts of the invention may include, without limitation, salts of alkali metals, e.g., sodium, potassium; salts of alkaline earth metals, e.g., calcium, magnesium, and barium; and salts of organic bases, e.g., amine bases and inorganic bases. Exemplary salts are found in Remington's Pharmaceutical Sciences, 17th ed., Mack Publishing Company, Easton, Pa., 1985, p. 1418, Berge et al., J. Pharmaceutical Sciences 66:1 (1977), and Pharmaceutical Salts: Properties, Selection, and Use, (Eds. P. H. Stahl and C. G. Wermuth), Wiley-VCH, 2008, each of which is incorporated herein by reference in its entirety.

The compositions of the invention may include a cysteamine precursor, or a salt thereof, in a component of a gastroretentive or mixed formulation to achieve plasma concentrations of cysteamine in the therapeutic range within the first 4 hours following administration, preferably within the first 2 hours following administration, and most preferably within the first hour. The cysteamine plasma concentration preferably remains in the therapeutic range for at least 5 hours, preferably 6 hours, more preferably 8 hours, 10 hours or longer. The formulation may include a thiol cysteamine precursor which can be enzymatically degraded to produce cysteamine, such as pantetheine, or a compound which can be degraded to pantetheine (and thence cysteamine) in the gastrointestinal tract, such as 4-phosphopantetheine, dephospho-coenzyme A or coenzyme A, or derivatives or prodrugs thereof that can be degraded to pantetheine in the gastrointestinal tract (and then to cysteamine). Alternatively, the cysteamine precursor may be formed by reacting cysteamine, or a compound which can be degraded to produce cysteamine, with another thiol-containing organosulfur compound to form a disulfide compound. A disulfide cysteamine precursor, or a salt thereof, may be formed by reacting cysteamine with a thiol cysteamine precursor such as pantetheine, 4-phosphopantetheine, dephospho-coenzyme A, coenzyme A or N-acetylcysteamine, or by reacting cysteamine with other thiols including N-acetylcysteine (NAC), N-acetylcysteine amide, N-acetylcysteine ethyl ester, homocysteine, glutathione (GSH), allyl mercaptan, furfuryl mercaptan, benzyl mercaptan, thioterpineol (grapefruit mercaptan), 3-mercaptopyruvate, L-cysteine, L-cysteine ethyl ester, L-cysteine methyl ester, thiocysteine, cysteinylglycine, gamma-glutamylcysteine, gamma-glutamylcysteine ethyl ester, glutathione monoethyl ester, glutathione diethyl ester, mercaptoethylgluconamide, thiosalicylic acid, tiopronin or diethyldithiocarbamic acid. Thiol cysteamine precursors, or cysteamine, may also be reacted with dithiols such as dihydrolipoic acid, meso-2,3-dimercaptosuccinic acid (DMSA), 2,3-dimercaptopropanesulfonic acid (DMPS), 2,3-dimercapto-1-propanol (dimercaprol), bucillamine or N,N′-bis(2-mercaptoethyl)isophthalamide (BDTH₂) to form disulfide cysteamine precursors.

The disulfides formed may delay the release of cysteamine in the stomach and/or facilitate its in vivo generation and absorption in the small intestine, depending on the properties of the cysteamine precursor used (e.g. the number of degradative steps required to form cysteamine). The stomach is generally a more oxidizing and more acidic environment than the small intestine. When the gastric contents pass into the duodenum they mix with pancreatic juice, which contains bicarbonate that neutralizes stomach acid, and with bile, which contains the physiologic reducing agent glutathione at millimolar concentrations, as well as related thiols including cysteine. Consequently, disulfides tend to remain oxidized in the stomach and are more likely to be reduced, or to participate in disulfide exchange reactions with thiols, in the small intestine. Disulfide exchange reactions are generally catalyzed by the thiolate ion, which is much more nucleophilic than the thiol form; thiolate ion formation is not favored in the acidic environment of the stomach.

For instance pantetheine, a thiol cysteamine precursor, may form a homodimeric disulfide where two pantetheines are covalently linked to form a pantethine (a disulfide cysteamine precursor). In some preferred embodiments, the cysteamine precursor provides more than one cysteamine, as provided by, for example, the mixed cysteamine disulfides formed by joining cysteamine with either pantetheine, 4-phosphopantetheine, dephospho-coenzyme A or coenzyme A, or by the corresponding mixed pantetheine disulfides formed by oxidizing pantetheine with either 4-phosphopantetheine, dephospho-coenzyme A or coenzyme A, or a suitable prodrug or analog convertible to the parent compound in the gastrointestinal tract. Also, 4-phosphopantetheine can be disulfide bonded to dephospho-coenzyme A or coenzyme A, or dephospho-coenzyme A can be disulfide bonded to coenzyme A to make cysteamine precursors capable of yielding two cysteamines in vivo. In some embodiments, the reactive thiol group of cysteamine or an organosulfur may be modified to include a substituent such as an acetyl group, ester group, glutamyl, succinyl, phenylalanyl, polyethylene glycol (PEG), and/or a folate.

In preferred embodiments, the composition of the invention may include a pantetheine, a disulfide containing pantetheine, or a salt thereof, in a component of the gastroretentive formulation and/or a component of a mixed formulation to sustain elevated blood levels of cysteamine for 5-10 hours after administration or longer. The composition may be a cysteamine precursor that requires chemical reduction or enzymatic conversion of the parent compound into at least one cysteamine, thereby delaying the release of cysteamine. The formulation may include pantetheine, or a compound which can be degraded to pantetheine in the gastrointestinal tract (e.g. 4-phosphopantetheine, dephospho-coenzyme A or coenzyme A; collectively pantetheine precursors), in which the thiol group of pantetheine, or a pantetheine precursor, is reacted with a thiol group of another organosulfur compound to form a disulfide compound. Since pantetheinase is expressed at higher levels in the intestine than in the stomach, and the lumen of the small intestine is a more reducing environment than the stomach, the pantetheine component of a disulfide cysteamine precursor may be converted to cysteamine, and subsequently absorbed, in the small intestine. For instance, pantetheine may form a homodimeric disulfide in which two pantetheines are covalently linked to form a pantethine. Pantetheine-containing cysteamine precursors may also include pantetheine mixed disulfides, where the pantetheine thiol reacts with a thiol group to form a disulfide. In preferred embodiments, the pantetheine precursor provides more than one cysteamine, as provided, for example, by the mixed disulfide formed from cysteamine and pantetheine, which when reduced and subsequently cleaved by pantetheinase yields 2 cysteamines and one pantothenic acid; or by the mixed disulfide pantetheine-coenzyme A, which when reduced and subsequently degraded and then cleaved by pantetheinase yields 2 cysteamines, 2 pantothenic acids, and ADP. In some embodiments, the reactive thiol group of pantetheine or an organosulfur compound may be modified to include a substituent such as an acetyl group, methyl ester, ethyl ester, glutamyl, succinyl, phenylalanyl, polyethylene glycol (PEG), and/or a folate.

The distinction between cysteamine precursors requiring pantetheinase cleavage to generate cysteamine vs. cysteamine precursors requiring only chemical reduction to generate cysteamine (cysteamine mixed disulfides) is significant because the kinetics of conversion of the precursor compound to cysteamine are generally more rapid with the second category, provided an adequately reducing environment exists (or can be created pharmacologically) in the intestine. A further distinction can be made between cysteamine precursors requiring reduction followed by pantetheinase cleavage (e.g. pantethine) vs. cysteamine precursors requiring first reduction then degradation to pantetheine then pantetheinase cleavage (e.g. 4-phosphopantetheine, dephospho-coenzyme A or co-enzyme A containing disulfides). The additional degradation step(s) required by the latter class of disulfide cysteamine precursors slows and extends the period of cysteamine production over a longer time period.

The compounds of the present invention can be prepared in a variety of ways known to one of ordinary skill in the art of chemical synthesis. Methods for preparing thiols, including cysteamine, pantetheine, 4-phosphopantetheine, dephospho-coenzyme A or coenzyme A and other thiols are well known in the art. Coenzyme A, pantethine, N-acetylcysteamine and glutathione are available commercially as dietary supplements.

The cysteamine precursor compounds utilized in the methods of the invention can be prepared as described in U.S. Ser. No. 16/648,725, filed Mar. 19, 2020, and incorporated herein by reference in its entirety.

Synthesis of Cysteamine Precursors

The present compounds, including both thiol and disulfide cysteamine precursors can be prepared from readily available starting materials using methods and procedures known in the art, such as those described by Mandel et al., Organic Letters, 6:4801 (2004). Methods for manufacturing pantethine are described in U.S. Pat. Nos. 3,300,508 and 4,060,551, each of which is incorporated herein by reference. Methods for converting liquid pantetheine to a solid form are disclosed in Japanese Patents Publication Nos. JP-A-S50-88215 and JP-A-S55-38344. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one of ordinary skill in the art by routine optimization procedures.

In preferred embodiments the composition of the invention includes one or more disulfide cysteamine precursors. Disulfides, being oxidized forms of thiols, are readily formed from constituent thiols without expensive reagents or equipment. Further, disulfides are not subject to the oxidation that can limit the long term stability of thiol compounds exposed to air. Thus with respect to manufacturing, cost, storage cost, shipping and patient convenience (i.e. long shelf life), disulfide forms of cysteamine precursors are preferable to thiol forms.

In some embodiments, mixed disulfide cysteamine precursors are synthesized by joining two different thiols, forming three reaction products: thiols A and B can join to form disulfides A-A, A-B and B-B. For example, disulfides formed by reacting cysteamine with pantetheine include: cysteamine-cysteamine (referred to as cystamine), cysteamine-pantetheine and pantetheine-pantetheine (referred to as pantethine). All three compounds are useful in providing cysteamine, and in fact the dissimilar steps involved in converting each compound to cysteamine can be pharmacologically beneficial by expanding the period of time over which cysteamine is generated in vivo by disulfide bond reduction or by a combination of reduction and enzymatic degradation steps. Thus the co-formulation of all three oxidation products without purification (except to remove unreacted thiols and/or unwanted impurities such as solvent) may be pharmacologically useful. This is particularly so when the two reacted thiols are each convertible into cysteamine (e.g. pantetheine, 4-phosphopantetheine, dephospho-coenzyme A, coenzyme A, N-acetylcysteine or suitable analogs and prodrugs), or when cysteamine itself is reacted with a thiol convertible into cysteamine. Consequently, in certain embodiments all three disulfides formed by reacting two different thiols, each convertible to cysteamine (or one of which is cysteamine), are co-formulated in a single composition. This method of synthesis and formulation does not require the more complex synthetic steps, or the post-synthesis purification steps required to separate a mixed disulfide from the two homodimeric disulfides which are created simultaneously in the oxidation reaction. (Unreacted thiols and other impurities must of course be removed before formulating a pharmaceutical composition.)

The advantages of manufacturing and co-formulating a mixture of three disulfides are not as fully realized in the case of disulfide cysteamine precursors made by reacting a thiol convertible to cysteamine with a second thiol not convertible to cysteamine. For example the three disulfides formed by reacting pantetheine with N-acetylcysteine (NAC) are: pantetheine-pantetheine (pantethine), pantetheine-NAC and NAC-NAC. The first two compounds are cysteamine precursors, the third (NAC-NAC) is not. However, NAC-NAC may nevertheless have beneficial pharmacological properties with respect to modulating the intestinal redox environment, or beneficial medical properties as a consequence of providing, upon chemical reduction, two NAC molecules. Thus in certain embodiments all three disulfide products formed by reacting cysteamine or a thiol convertible into cysteamine in vivo with a second thiol not convertible into cysteamine in vivo are co-formulated in a single composition.

The expected ratio of reaction products when two different thiols are oxidized depends on the molar ratio of the two thiols, the absolute concentration of the two thiols, pH, and/or the chemical environment around the sulfhydryl group of each thiol. If the ratio of thiol A to thiol B is 1:1 the expected molar ratio of the reaction products A-A, B-B, A-B is about: 1:1:2. (Deviations from the expected ratio may occur as a result of differences in the chemical bonds adjacent to the thiol that may affect, for example, the kinetics of disulfide bond formation, which may be influenced by the electronegativity of the atom bound to the sulfhydryl. Any deviation can be predicted or measured using methods known in the art.) The ratio of reaction products can be altered by changing the molar ratio of the two thiols. For example to increase the proportion of A-A and A-B relative to B-B the molar concentration of thiol A may be increased relative to that of thiol B. When reacting two thiols, one of which is cysteamine or a compound degradable to cysteamine (thiol A) and the other a thiol not degradable to cysteamine (thiol B), the molar concentration of the first thiol may be increased relative to that of the second thiol so as to increase the proportion of cysteamine precursors produced. For example reacting thiols A and B in a molar ratio of 2:1 increases the proportion of A-A and A-B (both cysteamine precursors) relative to B-B (not a cysteamine precursor).

In certain embodiments the oxidation of two unlike thiols can be promoted, and/or the mix of reaction products altered, by inclusion of a catalyst (reviewed in Musiejuk and Witt (2015)). For example an oxidant like hydrogen peroxide or dimethyl sulfoxide (DMSO) can be added, or a metal such as copper, manganese or telluride, or iodine, diethylazodicarboxylate (or related compounds), or dichlorodicyanoquinone (DDQ). Optimal performance of the catalyst can be achieved by empirically determining the best solvent system, catalyst concentration and reaction conditions.

In other embodiments an asymmetric disulfide can be produced via a thiol-disulfided exchange reaction between a thiol and a symmetrical disulfide. This type of reaction, like the oxidation of two dissimilar thiols, provides a mixture of all possible products (symmetrical and unsymmetrical disulfides). However, by providing a molar excess of the symmetrical disulfide over the thiol, formation of the unsymmetrical disulfide is favored, and may even be the major reaction product under optimized conditions. The method works with cysteamine as the thiol and pantethine as the disulfide, and with pantetheine as the thiol and cystamine as the disulfide. In preferred embodiments of the thiol:disulfide exchange reaction the molar ratio of thiol to disulfide (e.g. cysteamine:pantetheine) is between 2:1 and 4:1, between 2.5:1 and 3.5:1, between 2.7:1 and 3.3:1. In certain embodiments the solvent is methanol and the reaction time is between 1-20 hours, or between 1-12 hours, or between 1-6 hours. In certain embodiments the product of the thiol:disulfide exchange reaction (e.g. compound 1) is subsequently precipitated.

Alternatively, in another embodiment the ratio of cysteamine precursors used in a pharmaceutical composition may be adjusted by combining the three reaction products of a mixed disulfide oxidation reaction with a pure disulfide. For example, if the thiols cysteamine (C) and pantetheine (P), are oxidized in a 1:1 molar ratio they will combine to form 3 products: C-C, P-P and C-P in a ratio of approximately 1:1:2. Pure pantethine (P-P) can be added to the mixture in any desired amount to prolong the in vivo cysteamine-generating properties of the mixture. Doubling the starting amount of pantethine would yield a ratio of 1:2:2. Adding four times the starting amount of pantethine would yield a ratio of 1:2:5.

Two independently generated mixed disulfide reaction products may also be combined to achieve novel ratios of cysteamine precursors. For example, if the cysteamine-pantetheine reaction products (C-C, P-P and C-P) are combined with an equimolar quantity of reaction products from an N-acetylcysteine (NAC)-cysteamine (C) oxidation reaction (C-C, NAC-NAC and C-NAC in a ratio of 1:1:2), the mixture will contain five compounds, one of which, NAC-NAC, can not be converted to cysteamine. The other four disulfides, P-P, C-C, C-P, C-NAC are present in a molar ratio of approximately 1:2:2:2. Optionally, pantetheine may be added to make the ratio, for example, 2:2:2:2 (more simply expressed as 1:1:1:1) or added in greater quantity to make the ratio 1:1:1:5. Thus the molar ratio of disulfides in a pharmaceutical composition can be controlled by a variety of methods. In another example, the cysteamine-pantetheine reaction products (C-C, P-P and C-P) may be combined with an equimolar quantity of reaction products from a 4-phosphopantetheine (4P)-cysteamine (C) oxidation reaction (namely C-C, 4P-4P and C-4P in a ratio of 1:1:2), to produce a mixture of five disulfides in a ratio 1:1:1:2:2.

In summary, when oxidizing one thiol to make a cysteamine precursor disulfide there is only one product (e.g. pantetheine+pantetheine=pantethine). When oxidizing two thiols there are three products, either two or three of which are cysteamine precursors, depending on whether one or both of the thiols is degradable to cysteamine, or is cysteamine. Mixtures of cysteamine precursors are most easily made by combining the products of these two types of reactions. Mixtures may include various molar ratios of pure disulfide or three-component disulfide mixtures. However, heterodimeric cysteamine precursors may also be used in pure form, after purification, or combined with other homo- or heterodimeric cysteamine precursors.

Alternatively, by using more sophisticated chemical methods specific mixed disulfides (also called unsymmetrical disulfides) may be selectively synthesized (e.g. cysteamine and pantetheine can be combined to form substantially only the disulfide cysteamine-pantetheine). These methods employ a wide range of sulfur-protecting groups and strategies for their removal. The most widely used approach entails substitution of a sulfenyl derivative with a thiol or its derivative. Commonly utilized sulfenyl derivatives include: sulfenyl chlorides, S-alkyl thiosulfates and S-aryl thiosulfates (Bunte salts), S-(alkylsulfanyl)isothioureas, benzothiazol-2-yl disulfides, benzotriazolyl sulfides, dithioperoxyesters, (alkylsulfanyl)dialkylsulfonium salts, 2-pyridyl disulfides and derivatives, N-alkyltetrazolyl disulfides, sulfenamides, sulfenyldimesylamines, sulfenyl thiocyanates, 4-nitroarenesulfenanilides, thiolsulfinates and thiolsulfonates, sulfanylsulfinamidines, thionitrites, sulfenyl thiocarbonates, thioimides, thiophosphonium salts and 5,5-dimethyl-2-thioxo-1,3,2-dioxaphosphorinan-2-yl disulfides. Still other procedures involve: reaction of a thiol with a sulfinylbenzimidazole, rhodium-catalyzed disulfide exchange, electrochemical methods, and the use of diethyl azodicarboxylate. These and other methods are reviewed by Musiejuk, M. and D. Witt. Organic Preparations and Procedures International 47:95 (2015). Thus with only modest effort a specific mixed (unsymmetrical) disulfide of interest can be made. Examples 1 and 2 provide synthetic procedures for mixed disulfides of the invention.

In still other embodiments a mixed disulfide can be synthesized from a symmetric disulfide by preferentially coupling a substituent (e.g. an acyl group) to one end of the symmetric disulfide (i.e. hemi-acylation). For example, since cysteamine and pantetheine differ by a pantothenate moiety, the disulfide cystamine can be hemi-acylated by pantothenate to produce cysteamine-pantetheine disulfide. When the concentration of reactants is optimized, and coupling agents added to facilitate the acylation, this procedure can produce yields of greater than 95 percent. Cystamine is an attractive starting point for creating assymetric disulfides because it contains reactive amino groups at both ends. In certain embodiments the molar ratio of the acyl group to the disulfide is between 1:2 and 1:4. In certain embodiments the acylation reaction is facilitated by addition of N,N′-dicyclohexylcarbodiimide (DCC) at a molar ratio of DCC:acyl group between 3:1 and 5:1. In certain embodiments the acylation reaction is facilitated by addition of 1-Hydroxybenzotriazole (HOBt) at a molar ratio of HOBt:acyl group between 1:1 and 1:3.

Stereochemistry

Some of the compounds of the invention exist in more than one enantiomeric form. In particular pantetheine, 4-phosphopantetheine, dephospho-coenzyme A and coenzyme A contain a chiral carbon in the pantothenoyl moiety. Thus each of these compounds can exist as the D- or L-enantiomer, or as a racemic mixture of the two with respect to the pentaethenyl group. However, human pantetheinases (encoded by the VNN1 and VNN2 genes) are specific for D-pantetheine. (Bellussi et al., Physiological Chemistry and Physics 6:505 (1974)). Thus only D-pantetheine (and not L-pantetheine) is a cysteamine precursor, and accordingly the present invention concerns only D-pantetheine, and only the D-enantiomers of 4-phosphopantetheine, dephospho-coenzyme A and coenzyme A and any analogs or prodrugs convertible to those compounds in the gastrointestinal tract. Likewise, all disulfides that contain a pantetheine, 4-phosphopantetheine, dephospho-coenzyme A and coenzyme A, or any suitable analog or prodrug, only employ the D-enantiomer.

The L-enantiomer of amino acids and amino acid derivatives is preferred. Thus “cysteine” herein refers to L-cysteine, homocysteine to L-homocysteine, and cysteine derivatives such as N-acetylcysteine, N-acetylcysteine amide, N-acetylcysteine ethyl ester, cysteine methyl ester, cysteine ethyl ester, cysteinylglycine and gamma glutamyl cysteine are all formed using the L-enantiomer of cysteine.

For dihydrolipoic acid the R enantiomer is preferred, as that is the enantiomer made in the human body. In general, for compounds that are normally present in the human body or that are present in foods the naturally occurring enantiomer is preferred.

### Formulations

When employed as pharmaceuticals, cysteamine precursors, or a pharmaceutically acceptable salt, or prodrug thereof can be administered in the form of pharmaceutical compositions. These compositions can be prepared in a variety of ways well known in the pharmaceutical art, and can be made so as to release drug in specific segments of the gastrointestinal tract at controlled times by a variety of excipients and formulation technologies. For example, formulations may be tailored to address a specific disease, to achieve blood levels of cysteamine required to achieve therapeutic efficacy, to enable a desired duration of drug effect, and to provide a set of compositions with varying drug release characteristics that can be administered in different combinations to account for inter-patient variation in cysteamine metabolism. Administration is primarily by the oral route and may be supplemented by suppositories. Cysteamine precursors may also be co-formulated with agents that enhance in vivo cysteamine generation or absorption, including, for example, reducing agents, buffers, pantetheinase inducers or inducers of cysteamine uptake by intestinal epithelial cells.

The pharmaceutical composition can contain one or more pharmaceutically acceptable carriers. In making a pharmaceutical composition for use in a method of the invention, the cysteamine precursor, pharmaceutically acceptable salt, solvate, or prodrug thereof is typically mixed with an excipient, diluted by an excipient or enclosed within such a carrier in the form of, for example, a capsule, tablet, sachet, paper, vial or other container. The active component of the invention can be administered alone, or in a mixture, in the presence of a pharmaceutically acceptable excipient or carrier. The excipient or carrier is selected on the basis of the mode and route of administration, the region of the gastrointestinal tract targeted for drug release, and the intended time profile of drug release. When the excipient serves as a diluent, it can be a solid, semisolid, or liquid material (e.g., normal saline), which acts as a vehicle, carrier, matrix or other medium for the active ingredient. Thus, the compositions can be in the form of tablets, powders, granules, lozenges, sachets, cachets, elixirs, suspensions, emulsions, solutions, syrups, and soft and hard gelatin capsules. As is known in the art, the type and amount of excipients vary depending upon the intended drug release characteristics. The resulting compositions can include additional agents, such as preservatives or coatings.

Suitable pharmaceutical carriers, as well as pharmaceutical necessities for use in pharmaceutical formulations, are described in Remington: The Science and Practice of Pharmacy, 21st Ed., Gennaro, Ed., Lippencott Williams & Wilkins (2005), a well-known reference text in this field, and in the USP/NF (United States Pharmacopeia and the National Formulary) or corresponding European or Japanese reference documents. Examples of suitable excipients are lactose, dextrose, sucrose, sorbitol, mannitol, starches, gum acacia, calcium carbonate, calcium phosphate, alginates, tragacanth, gelatin, calcium silicate, microcrystalline cellulose, cellulose derivatives, polyvinylpyrrolidone, poly(lactic-co-glycolic acid) (PLGA), cellulose, water, syrup, methyl cellulose, vegetable oils, polyethylene glycol, hydrophobic inert matrix, carbomer, hypromellose, gelucire 43/01, docusate sodium, and white wax. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients and details of their use are described in Handbook of Pharmaceutical Excipients, 6th Edition, Rowe et al., Eds., Pharmaceutical Press (2009).

The pharmaceutical composition can include cysteamine precursor salts, optionally co-formulated or co-administered with other agents that enhance the in vivo degradation of cysteamine precursors to cysteamine or enhance the intestinal absorption of cysteamine. The pharmaceutical composition may also include other therapeutic agents that complement the pharmacological effects of cysteamine in targeted diseases. Exemplary enhancers of in vivo cysteamine production or absorption, and exemplary therapeutic agents that may be included in the compositions described herein are provided herein.

The compositions of the invention may contain a single active component (i.e. a single cysteamine precursor), or a combination of a first and a second active component in a single unit dosage form, or a combination of a first, second, third and, optionally, a fourth active and optionally a fifth component in a single unit dosage form. In compositions with two active components both components may be cysteamine precursors or one component may be an enhancer of in vivo cysteamine production (e.g. a reducing agent that promotes reduction of disulfide cysteamine precursors, or an agent that induces increased intestinal expression of pantetheinase) or an enhancer of intestinal absorption of cysteamine (e.g. an agent that induces increased expression of one or more organic cation transporters, such as OCT1, OCT2 or OCT3). In compositions with three or four active components all components may be cysteamine precursors or one or two components may be enhancers of in vivo cysteamine production and/or intestinal absorption. In compositions with two or more cysteamine precursors the types of cysteamine precursors are selected to achieve in vivo cysteamine production over a sustained time period. For example a mixed disulfide cysteamine precursor, which only requires disulfide bond reduction to generate one cysteamine, and will therefore start generating cysteamine shortly after reaching a region of the gastrointestinal tract with a redox environment conducive to disulfide bond reduction, can be mixed with pantetheine, or with a pantetheine disulfide, which requires both disulfide bond reduction and pantetheinase cleavage to yield cysteamine, and optionally also combined with a compound degradable to pantetheine in the gut, or a disulfide containing such a compound, which requires additional steps to generate pantetheine and thence cysteamine. Compounds degradable to pantetheine in the gut include 4-phosphopantetheine, dephospho-coenzyme A, coenzyme A and suitable analogs and derivatives. The time course of in vivo cysteamine production will vary according to the number of degradative steps between the cysteamine precursor and cysteamine. In some embodiments compositions containing multiple cysteamine precursors are formulated as a powder, as granules or as a liquid—i.e. formulation types that can accommodate large quantities of drug substance.

The pharmaceutical composition may also include one or more agents that enhance the performance of the formulation. For example a gastroretentive composition may include a compound that slows gastric emptying in order to prolong the residence of the composition in the stomach.

In compositions with two cysteamine precursor components the first and second components may be present at a ratio of, for example, about 1:1.5 to about 1:4. In compositions with three cysteamine precursor components the first, second and third components may be present at a ratio of, for example, between about 1:1:2 to about 1:4:4. In compositions with four active components the first through fourth active components may be present at a ratio of, for example, about 1:1:1:2 to about 1:2:5:5. In compositions with five active components the first through fifth active components may be present at a ratio of, for example, about 1:1:2:2:2 to about 1:1:2:5:5:8.

In some embodiments compositions that contain two or more cysteamine precursors include one precursor selected for rapid in vivo cysteamine production (e.g. simply requiring disulfide bond reduction) and a second precursor selected for intermediate or slower in vivo conversion to cysteamine e.g. requiring chemical reduction and at least one enzymatic degradative step). In some embodiments a pharmaceutical composition containing two or more cysteamine precursors at least one precursor is a cysteamine mixed disulfide, which can yield cysteamine upon disulfide bond reduction. In additional related embodiments at least one additional component is a disulfide containing pantetheine or a compound degradable to pantetheine in the gastrointestinal tract.

The compositions can be formulated in a solid unit dosage form (e.g. a tablet or capsule), each dosage containing, e.g., 50-800 mg of the active ingredient of the first component. For example, the dosages can contain from about 50 mg to about 800 mg, from about 50 mg to about 700 mg, from about 50 mg to about 600 mg, from about 50 mg to about 500 mg; from about 75 mg to about 800 mg, from about 75 mg to about 700 mg, from about 75 mg to about 600 mg, from about 75 mg to about 500 mg; from about 100 mg to about 800 mg, from about 100 mg to about 700 mg, from about 100 mg to about 600 mg, from about 100 mg to about 500 mg; from about 250 mg to about 800 mg, from about 250 mg to about 700 mg, from about 250 mg to about 600 mg, from about 250 mg to about 500 mg; from about 400 mg to about 800 mg, from about 400 mg to about 700 mg, from about 400 mg to about 600 mg; from about 450 mg to about 700 mg, from about 450 mg to about 600 mg of the active ingredient of a first component.

In alternative embodiments compositions can be formulated in a liquid or powdered unit dosage form, each dosage unit containing from about 250 mg to about 10,000 mg of cysteamine precursor. For example, the dosages can contain from about 250 mg to about 10,000 mg, from about 250 mg to about 8,000 mg, from about 250 mg to about 6,000 mg, from about 250 mg to about 5,000 mg; from about 500 mg to about 10,000 mg, from about 500 mg to about 8,000 mg, from about 500 mg to about 6,000 mg, from about 500 mg to about 5,000 mg; from about 750 mg to about 10,000 mg, from about 750 mg to about 8,000 mg, from about 750 mg to about 6,000 mg, from about 750 mg to about 5,000 mg; from about 1,250 mg to about 10,000 mg, from about 1,250 mg to about 8,000 mg, from about 1,250 mg to about 6,000 mg, from about 1,250 mg to about 5,000 mg; from about 2,000 mg to about 10,000 mg, from about 2,000 mg to about 8,000 mg, from about 2,000 mg to about 6,000 mg; from about 2,000 mg to about 5,000 mg, from about 3,000 mg to about 6,000 mg of the active ingredient of a first component.

In compositions with a first and second cysteamine precursor component the amount of the second active component in a solid unit dosage form can vary, e.g., from 50-700 mg. For example, the dosage can contain from about 50 mg to about 700 mg, from about 50 mg to about 600 mg, from about 50 mg to about 500 mg, from about 50 mg to about 450 mg; from about 75 mg to about 700 mg, from about 75 mg to about 600 mg; from about 100 mg to about 700 mg; from about 100 mg to about 600 mg, from about 100 mg to about 500 mg, from about 100 mg to about 400 mg; from about 250 mg to about 700 mg, from about 250 mg to about 600 mg, from about 250 mg to about 500 mg, from about 250 mg from to about 400 mg; from about 400 mg to about 700 mg, from about 400 mg to about 600 mg, from about 400 mg to about 500 mg, from about 450 mg to about 700 mg; from about 450 mg to about 600 mg, from about 450 mg to about 500 mg. In a composition with a cysteamine precursor as the first active component and an enhancer of in vivo cysteamine generation as the second active component the amount of the second active component in a unit dosage form can vary, e.g. from 0.1 mg-400 mg.

In alternative embodiments including a first and second cysteamine precursor component the amount of the second active component in a liquid or powdered unit dosage form can vary, e.g., from about 250 mg to about 6,000 mg. For example, the dosage can contain from about 250 mg to about 6,000 mg per dose, from about 250 mg to about 5,000 mg, from about 250 mg to about 4,000 mg, from about 250 mg to about 3,000 mg, from about 250 mg to about 2,000 mg; from about 500 mg to about 6,000 mg, from about 500 mg to about 5,000 mg, from about 500 mg to about 4,000 mg, from about 500 mg to about 3,000 mg; from about 750 mg to about 6,000 mg, from about 750 mg to about 5,000 mg, from about 750 mg to about 4,000 mg, from about 750 mg to about 3,000 mg; from about 1,250 mg to about 6,000 mg, from about 1,250 mg to about 5,000 mg, from about 1,250 mg to about 4,000 mg, from about 1,250 mg to about 3,000 mg; from about 2,000 mg to about 6,000 mg, from about 2,000 mg to about 5,000 mg, from about 2,000 mg to about 4,000 mg; from about 2,000 mg to about 3,000 mg, from about 2,500 mg to about 5,000 mg of the active ingredient of a second component

In solid compositions with a third, or third and fourth cysteamine precursor component the unit dosages can contain from about 50 mg to about 400 mg of each of the third and, if present, fourth active components. For example, the dosages can contain from about 50 mg to about 400 mg, from about 50 mg to about 350 mg, from about 50 mg to about 300 mg, from about 50 mg to about 250 mg; from about 75 mg to about 400 mg, from about 75 mg to about 350 mg, from about 75 mg to about 300 mg, from about 75 mg to about 250 mg; from about 100 mg to about 400 mg, from about 100 mg to about 350 mg, from about 100 mg to about 300 mg, from about 100 mg to about 250 mg; from about 250 mg to about 400 mg, from about 250 mg to about 350 mg or from about 250 mg to about 300 mg. In compositions with five active components the unit dosages of the five components can range from about 50 mg to about 300 mg. In a composition with an enhancer of in vivo cysteamine generation as the fourth, and optionally also the third active component the amount of the fourth, and optionally the third active components in a unit dosage form can vary, e.g. from 0.1 mg-400 mg.

In alternative embodiments including a third, or a third and fourth cysteamine precursor component in a liquid or powdered unit dosage form the unit dosages of the third and optionally fourth active component can vary, e.g., from about 250 mg to about 4,000 mg. For example, the dosage can contain from about 250 mg to about 4,000 mg per dose, from about 250 mg to about 3,000 mg, from about 250 mg to about 2,000 mg, from about 250 mg to about 1,000 mg, from about 500 mg to about 4,000 mg, from about 500 mg to about 3,000 mg, from about 500 mg to about 2,000 mg, from about 500 mg to about 1,000 mg; from about 750 mg to about 4,000 mg, from about 750 mg to about 3,000 mg, from about 750 mg to about 2,000 mg, from about 750 mg to about 1,000 mg; from about 1,000 mg to about 4,000 mg, from about 1,000 mg to about 3,000 mg, from about 1,000 mg to about 2,000 mg, from about 1,000 mg to about 1,500 mg; from about 1,500 mg to about 4,000 mg, from about 1,500 mg to about 3,000 mg, from about 1,500 mg to about 2,000 mg; from about 2,000 mg to about 4,000 mg, from about 2,000 mg to about 3,000 mg of the active ingredient of a third and optionally fourth active component

The pharmaceutical compositions can be formulated so as to provide immediate, delayed, gastroretentive, sustained or colonic release (collectively referred to as controlled release) of the active component after administration to the patient by employing procedures known in the art.

For preparing solid compositions such as tablets, the active ingredient or ingredients (e,g. several cysteamine precursors) may be mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets, capsules or microparticles. This solid bulk formulation is then subdivided into unit dosage forms of the type described above.

Alternatively two homogeneous batches of active ingredient(s) mixed with one or more pharmaceutical excipients may be prepared, each using a different concentration of active ingredient(s). The first mixture may then be used to form a core and the second mixture a shell around the core to form a composition with variable drug release characteristics. If the high concentration batch is located in the core and the lower concertation batch in the shell an initial moderate rate of drug release will be followed by a greater rate of drug release once the shell has substantially dissolved or eroded. In some embodiments a pharmaceutical composition contains a higher concentration of active ingredient(s) in the core than in the shell. The ratio of cysteamine precursor concentrations in the core:shell may, for example, range between about 1.5:1 to 4:1. The excipients may also differ in type or in concentration between the two batches, so as to influence the rate of drug release. In some embodiments the polymer(s) or other matrix-forming ingredients in the core release the active ingredient(s) more slowly than from the shell. In such embodiments a higher concentration of cysteamine precursor(s) in the core is partially or completely balanced by a slower rate of drug release, to extend the duration of cysteamine precursor release, and hence the duration of in vivo cysteamine generation, intestinal absorption and elevated blood levels. One or more coatings may be applied to the core before the shell layer is applied, and additional coatings may be applied to the shell to enable an efficient manufacturing process and/or to help provide desired pharmacological properties, including the timing and location of drug release in the gastrointestinal tract.

The pharmaceutical compositions of the invention include those formulated to release a mixture of cysteamine precursors which differ in the mechanism(s) or number of degradative steps leading to cysteamine production. Specifically, a mixture of two, three, four or five cysteamine precursors, each of which is one, two, three or more chemical and/or enzymatic degradative steps away from releasing cysteamine. For example the one step may be disulfide bond reduction (in the case of a cysteamine mixed disulfide) or pantetheinase cleavage (in the case of pantetheine). The two steps may be disulfide bond reduction followed by pantetheinase cleavage (in the case of a pantetheine disulfide) or phosphatase cleavage followed by pantetheinase cleavage (in the case of 4-phosphopantetheine). The three steps may be disulfide bond reduction preceded or followed by degradation to pantetheine (e.g. by a phosphatase), followed by pantetheinase cleavage (e.g. in the case of a 4-phosphopantetheine disulfide). The four steps may be disulfide bond reduction followed by two degradative steps to pantetheine (e.g. removal of the adenine nucleotide moiety by ecto-nucleotide diphosphatase followed by removal of the 4′ phosphate by a phosphatase), followed by pantetheinase cleavage (e.g. in the case of a coenzyme A or dephospho-coenzyme A disulfide). The purpose of combining cysteamine precursors that have different chemical and/or enzymatic degradative pathways to cysteamine is to extend the time during which cysteamine is produced in and absorbed from the gut, and consequently prolong the duration of therapeutically effective cysteamine blood levels. In some embodiments a pharmaceutical composition of the invention contains at least two cysteamine precursors, in further embodiments a pharmaceutical composition contains three cysteamine precursors.

The pharmaceutical compositions of the invention may be formulated for mixed release, meaning that one composition contains two drug release profiles. For example an immediate release formulation may be combined with a sustained release formulation. In such a composition, the first active component may be formulated for immediate release starting between about 5 minutes and about 30 minutes following ingestion. For example, the first active component may be released starting 5 minutes, 10 minutes, 15 minutes, 20 minutes, 25 minutes, 30 minutes, or 45 minutes after ingestion of the composition. The first active component is formulated such that cysteamine plasma concentrations in the therapeutic range are achieved between about 15 minutes and 3 hours following ingestion, preferably between 30 minutes and 2 hours. For example, therapeutic plasma cysteamine concentrations may be reached 0.5 hours, 1 hour, 2 hours, or 3 hours following ingestion of the composition. The type of cysteamine precursor used (e.g. thiol, cysteamine mixed disulfide, pantetheine disulfide, coenzyme A disulfide, N-acetylcysteamine disulfide, etc.) will influence the length of time to reach therapeutic blood concentrations of cysteamine, and the duration of time over which therapeutic blood concentrations are maintained.

In a composition with two, three, and optionally four or five active components (e.g. multiple cysteamine precursors and/or enhancers of in vivo cysteamine generation and absorption) each of the second, third, and/or fourth and/or fifth active components is formulated for controlled release from the composition starting between about 1 hour and about 8 hours following ingestion. A controlled release composition may include a delayed release and/or a sustained release formulation. For example, the second, third, and/or fourth active component may be released starting 1 hour, 1.5 hours, 2 hours, 3 hours, 4 hours, 5 hours, 6 hours, 7 hours, or 8 hours after ingestion of the composition. The second, third, and/or fourth active component is formulated such that the plasma concentration of cysteamine (which reflects the contributions of all active components) is maintained in the therapeutic range starting between about 30 minutes and 2 hours following ingestion and extending for between about 6 and 10 hours, more preferably extending for between 8 and 12 hours following ingestion, or for longer periods. For example, the plasma cysteamine concentration may be sustained in the therapeutic range for 6 hours, 8 hours, 10 hours, 12 hours, 15 hours, 20 hours, or 24 hours following ingestion of the active components of the composition. Depending on the age and size of the patient, the disease being treated, and the cysteamine metabolizing rate of the patient, two or more compositions may be needed to deliver enough cysteamine precursor to achieve therapeutic blood levels over multiple hours.

As an alternative or complement to pharmaceutical compositions comprising mixed formulations, in some embodiments compositions consisting of a single type of formulation may be produced. That is, time-based formulations such as immediate release or sustained release formulations, and anatomically-targeted formulations such as gastroretentive, delayed release and colon-directed formulations, may be prepared for administration as separate compositions. Formulating a collection of pharmaceutical compositions with different drug release properties (whether time-based or anatomically/physiologically-based) has certain advantages. For example, such compositions can be administered in different combinations and ratios to different patients to bring about blood cysteamine levels in the therapeutic range for an extended period of time. That is, a therapeutic regimen consisting of one, two, three or more compositions administered on a specific schedule can be tailored to the cysteamine generating, absorbing and metabolizing capacity of an individual patient. Since these capacities are known to vary among patients, the formulation of multiple homogeneous compositions containing different cysteamine precursors and different drug release properties, which can be combined in different ratios for different patients, addresses a known limitation of existing cysteamine formulations.

Preferably a combination of two or more pharmaceutical compositions can maintain cysteamine blood levels in the therapeutic range for at least hours 2-8 after ingestion, more preferably from hours 1-8 following ingestion, still more preferably from hours 2-10 and most preferably from hours 1-10, hours 1-12, hours 1-14, or longer. Separately formulated pharmaceutical compositions containing different cysteamine precursors with different drug release profiles provide the dosing flexibility needed to individualize dosing regimens to attain therapeutically effective cysteamine blood concentrations for prolonged periods.

It is well documented that gastric emptying time and large intestinal transit time vary considerably among healthy individuals (up to two-fold or more). The gut redox environment and levels of pantetheinase activity are also known to vary among individuals. These and other factors likely account for the wide inter-individual variation in plasma cysteamine levels observed following a cysteamine dose. For example in a study of immediate release cysteamine bitartrate pharmacokinetics in healthy volunteers the peak cysteamine blood level (Cmax) following a 600 mg oral dose, administered with a meal, varied over 8-fold, from 7 micromolar to 57.3 micromolar. (Dohil R. and P. Rioux, Clinical Pharmacology in Drug Development 2:178 (2013)). In the same study the Cmax following 600 mg of delayed release cysteamine bitartrate administered with a meal varied 12-fold, from 2.1 uM to 25.4 uM. Inter-patient variation in cysteamine plasma levels was less extreme when cysteamine was administered to fasting patients, but still up to four fold. (When cysteamine is dosed every six hours, as with Cystagon®, or even every 12 hours, as with Procysbi®, it is difficult to completely avoid meal times.)

Current methods of cysteamine formulation and administration provide only one tool to address inter-subject variability: raise or lower the dose. The cysteamine precursors, enhancers of in vivo cysteamine generation and absorption, drug formulation methods and drug administration methods of the invention provide multiple tools to achieve therapeutic blood cysteamine levels by tailoring compounds, dosage forms and dosing regimens to individual patients without incurring the unacceptable toxicity often associated with high Cmax or the inadequate therapeutic effect associated with prolonged blood levels below the therapeutic threshold.

Another advantage for separately formulated compositions is that they can be administered at different times with respect to meals. This is a useful option because different classes of cysteamine precursors and different types of formulations interact differently with meals. For example, a gastroretentive formulation should be administered with or shortly after a meal, preferably a nutrient rich meal to maximize the duration of gastric retention. Conversely, an immediate release formulation that contains a cysteamine mixed disulfide that can be rapidly converted to cysteamine by disulfide bond reduction should preferably not be administered with a large meal. Large meals interfere with absorption of cysteamine in some individuals, however meals are compatible with certain cysteamine precursors that produce little if any cysteamine in the stomach, e.g. pantetheine disulfides, which tend to be converted to cysteamine in the small intestine.

The individualized dosing regimens possible with the compounds and formulations of the invention are particularly useful because while extensive inter-individual variation in cysteamine intestinal absorption is well documented, it is equally well documented that intra-individual variation is moderate in comparison. That is, a given subject will absorb and metabolize a dose of cysteamine substantially similarly when administered on multiple occasions under similar circumstances. Thus a dosing regimen, once individualized to produce blood cysteamine levels in the therapeutic range for a specific patient, should be relatively stable and produce predictable results over time.

Sustained release formulations can be designed to release drugs over widely varying periods of time using methods known in the art. (Wen, H. and Park, K., editors: Oral Controlled Release Formulation Design and Drug Delivery: Theory to Practice, Wiley, 2010; Wells, J. I. and Rubinstein, M. H., editors: Pharmaceutical Technology: Controlled Drug Release, volumes I and II, Ellis and Horwood, 1991, and Gibson, M., editor: Pharmaceutical Preformulation and Formulation: A Practical Guide from Candidate Drug Selection to Commercial Dosage Form, 2^{nd} edition, Informa, 2009.)

Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals or granules, which contain the active ingredient(s) in a mixture with non-toxic pharmaceutically acceptable excipients. If formulated as a liquid, powder, crystals or granules the dose may be packaged in a manner that clearly demarcates a unit dose. For example a powder or granules or microparticles may be packaged in a sachet. A liquid may be packaged in a glass or plastic container.

Excipients are selected to provide acceptable organoleptic properties, to control drug release properties, to facilitate efficient manufacturing and to ensure long term stability of pharmaceutical compositions, among other considerations known to those skilled in the arts of pharmacology, pharmaceutics and drug manufacturing. The excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, preservatives, buffering agents, stabilizing agents and the like. Many of these excipients are sold by multiple excipient manufacturers in a variety of chemical forms, and/or can be used at different concentrations, and/or in different combinations with other excipients, with ensuing differences in performance characteristics. Specific excipients may accomplish more than one purpose in a formulation.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

One category of useful formulations mainly controls the rate of drug release (e.g. immediate and sustained release formulations), albeit with significant implications for where drug is released. A second category of useful formulations mainly controls the anatomical site of drug release (e.g. gastroretentive formulations for drug release in the stomach, colon-targeted formulations for the large intestine) albeit with implications for the timing of release. Enteric coated formulations have important elements of both: they are designed to remain intact in the acidic stomach environment, and often to dissolve in the more alkaline small intestine, which is a kind of anatomical targeting, yet they are often refer erred to as delayed release formulations, highlighting the time control element. However, colon targeted formulations may also have an enteric coating to prevent dissolution in the stomach, highlighting the complex relationship between anatomical targeting and control of the rate of drug release. Further, there is extensive overlap between the excipients used in time-based and anatomically- or physiologically-targeted formulations. These types of formulation can be combined in various ways to create a plurality of compositions with different drug release profiles, in both time and space. Such compositions can in turn be combined in different amounts and ratios to individualize therapeutic regiments to accommodate biochemical and physiologic variation among patients, as well as variation in disease type, extent and activity.

Gastroretentive Formulations

Gastroretentive formulations may be employed for release of a cysteamine precursor, or a salt thereof, from a composition of the invention in the stomach and to control the release of the active component(s) of the composition in the stomach over an extended period of time. In other words, since the point of a gastroretentive formulation is prolonged gastric residence, the accompanying excipients should provide for sustained release of active ingredients over the entire period of time that the gastroretentive dosage form is expected to remain in the stomach, and optionally longer, including the time of transit through the small intestine and into the colon. The gastroretention of active components of the invention may be achieved by various mechanisms, such as mucoadhesion, flotation, sedimentation, swelling and expansion, and/or by the simultaneous administration of pharmacological agents which delay gastric emptying. Excipients used in gastroretentive formulations, as well as the size and shape of pharmaceutical compositions, vary according to the mechanism of gastroretention.

Mucoadhesive/Bioadhesive Gastroretentive Formulations

Mucoadhesion relates to adhesion of a polymer utilized in the formulation to the gastrointestinal mucus layer until it is removed spontaneously from the surface as a result of ongoing mucus production. Bioadhesion, sometimes used interchangeably with mucoadhesion, also encompasses adhesion of a polymer or other component of a pharmaceutical composition to molecules on the surface of gastrointestinal epithelial cells. The purpose of mucoadhesion and bioadhesion is to increase the time that a pharmaceutical composition is in close proximity to gastrointestinal epithelial cells, including the cell types capable of cysteamine precursor cleavage (i.e. cells that express pantetheinase on their surface), and cysteamine uptake and transport into the circulation (e.g. cells expressing organic cation transporters). Mucoadhesive polymers can be used in formulating large dosage forms such as tablets or capsules and small dosage forms such as microparticles or microspheres. Various physiological factors such as peristalsis, mucin type, mucin turnover rate, gastrointestinal pH, fast/fed state and type of foods in the fed state affect the degree and persistence of mucoadhesion. The mechanism of mucoadhesion is thought to be through the formation of electrostatic and hydrogen bonds at the polymer-mucus boundary. Generally, mucoadhesion is achieved with polymers having affinity for gastrointestinal mucous and selected from synthetic or natural bioadhesive materials such as polyacrylic acids, methacrylic acids and derivatives of both, polybrene, polylysine, polycarbophils, carbomers, alginates, chitosan, cholestyramine, gums, lectins, polyethylene oxides, sucralfate, tragacanth, dextrins (e.g. hydroxypropyl beta-cyclodextrin), polyethylene glycol (PEG), gliadin, cellulose and cellulose derivatives such as hydroxypropyl methylcellulose (HPMC), or mixtures thereof. For example cross-linked acrylic and methacrylic acid copolymers available under the Trade Names CARBOPOL (e.g. Carbopol 974P and 971P) and POLYCARBOPHIL have been used in mucoadhesive formulations. (Hombach J. and A. Bernkop-Schnürch. Handbook of Experimental Pharmacology 197:251 (2010)). Other bioadhesive cationic polymers include acidic gelatin, polygalactosamine, poly-aminoacids such as polylysine, polyornithine, polyquaternary compounds, prolamine, polyimine, diethylaminoethyldextran (DEAE), DEAE-imine, polyvinylpyridine, polythiodiethylaminomethylethylene (PTDAE), polyhistidine, DEAE-methacrylate, DEAE-acrylamide, poly-p-aminostyrene, polyoxethane, Eudragit RL, Eudragit RS, GAFQUAT, polyamidoamines, cationic starches, DEAE-dextran, DEAE-cellulose and copolymethacrylates, including copolymers of HPMA, N-(2-hydroxypropyl)-methacrylamide (e.g. see U.S. Pat. No. 6,207,197).

Mucoadhesion is most effective when applied to small particles (e.g. microparticles). Mucoadhesive formulations may be combined with one or more other gastroretentive formulation methods described below, including floating formulations, expanding/swelling formulations, or any type of sustained release formulation.

Floating Gastroretentive Formulations

Flotation as a gastric retention mechanism is effective in formulations of the active component (e.g. cysteamine precursor) having a bulk density lower than that of gastric fluid and/or chyme (partially digested food in the stomach) so as to remain buoyant in the stomach. Generally a density of less than 1 gram per cubic centimeter is desirable, more preferably a density of less than 0.9 grams per cubic centimeter. Buoyancy can be achieved by (i) using low density materials, including lipids, (ii) pre-forming a gas bubble or bubbles in the center of a composition, or (iii) using effervescent excipients to generate gas bubbles in vivo. Pharmaceutical compositions of the latter type must be designed so that gas generated by the effervescent excipients remains in the composition and thereby contributes to its buoyancy. For example, the effervescent excipients can be embedded in a matrix of polymers to trap the bubbles in the composition. The latter type of buoyant formulations generally utilize matrices prepared with swellable polymers or polysaccharides and effervescent couples, e.g., sodium bicarbonate and citric or tartaric acid or matrices containing chambers of entrapped air or liquids that generate gas upon contact with liquid gastric contents at body temperature. Floating gastroretentive formulations have been reviewed extensively (e.g. Kotreka, U.K. Critical Reviews in Therapeutic Drug Carrier Systems, 28:47 (2011)).

Floating pharmaceutical compositions designed for gastric retention have been known in the art for some time. For example, U.S. Pat. Nos. 4,126,672, 4,140,755 and 4,167,558, each of which is incorporated herein by reference, describe a “hydrodynamically balanced” drug delivery system (HBS) in tablet form having a density less than that of gastric fluid (i.e. less than 1 gram per cubic centimeter). Consequently the composition floats on the stomach fluid or chyme, thereby avoiding ejection through the pylorus during muscular contractions of the stomach. Drug is continuously released from a cellulose-derived hydrocolloid such as methylcellulose, hydroxyalkylcelluloses (e.g. hydroxypropylcellulose, hydroxypropylmethylcellulose, hydroxyethylcellulose) or sodium carboxymethyl-cellulose, which, upon contact with gastric fluid, forms a water-impermeable barrier on the surface of the composition that gradually erodes, slowly releasing drug. A two-layered floating tablet, with an outer layer formulated for immediate release and an inner layer formulated for sustained release, is also disclosed in U.S. Pat. No. 4,140,755, incorporated herein by reference.

A similar hydrodynamically balanced floating formulation for sustained delivery of L-dopa and a decarboxylase inhibitor has also been described (see U.S. Pat. No. 4,424,235). Hydrocolloids, such as acacia, gum tragacanth, locust bean gum, guar gum, karaya gum, agar, pectin, carrageen, soluble and insoluble alginates, carboxypolymethylene, gelatin, casein, zein and bentonite can be useful in the preparation of floating formulations of the invention. The floating formulation can include up to about 60% of a fatty material or mixture of fatty materials selected from beeswax, cetyl alcohol, stearyl alcohol, glyceryl monostearate, hydrogenated castor oil and hydrogenated cottonseed oil (fats and oils have a lower density than gastric fluid). The floating formulations can promote sustained release of the cysteamine precursor and provide elevated plasma cysteamine levels for a longer period of time. The prolonged elevated plasma cysteamine levels permit less frequent dosing.

The floating compositions of the present invention may contain gas generating agents. Methods for formulating floating compositions using gas generating compounds are known in the art. For example, floating minicapsules containing sodium bicarbonate are described in U.S. Pat. No. 4,106,120. Similar floating granules based on gas generation are described in U.S. Pat. No. 4,844,905. Floating capsules have been described in U.S. Pat. No. 5,198,229.

Floating compositions may optionally contain an acid source and a gas-generating carbonate or bicarbonate agent, which together act as an effervescent couple, producing carbon dioxide gas which provides buoyancy to the formulation. Effervescent couples consisting of a soluble organic acid and an alkali metal carbonate salt form carbon dioxide when the mixture comes into contact with water or when the alkaline component comes into contact with an acidic liquid (e.g. gastric juice). Typical examples of acids used include citric acid, tartaric acid, malic acid, fumaric acid or adipic acid. Typical examples of gas generating alkalis used include sodium bicarbonate, sodium carbonate, sodium glycine carbonate, sodium sesquicarbonate, potassium carbonate, potassium bicarbonate, calcium carbonate, calcium bicarbonate, ammonium bicarbonate, sodium bisulfite, sodium metabisulfite, and the like. The gas generating agent interacts with an acid source triggered by contact with water, or with the hydrochloric acid in gastric juice, to generate carbon dioxide or sulfur dioxide that gets entrapped in the matrix of the composition and improves its floating characteristics. In one embodiment the gas generating agent is sodium bicarbonate and the acid source is citric acid.

The kinetics of flotation are important because if the composition is not lighter than gastric fluid and/or chyme soon after reaching the stomach there is a chance it will be rapidly expelled via the pylorus. Some compositions have a lower density than gastric fluid and chyme upon ingestion, such as compositions that contain pre-formed gas bubbles, or that contain low density materials such as lipids. For those floating compositions that must attain a density below that of gastric fluid and/or chyme after reaching the stomach (i.e. effervescent formulations) a density lower than 1 gram per cubic centimeter is preferably reached within 30 minutes, more preferably within 15 minutes, and most preferably within ten minutes after contact with gastric fluid. The duration of floating is also important and should be matched to the duration of drug release. That is, if the composition is designed to release drug over 6 hours it should also be able to float for six hours. Preferably a floating composition maintains a density less than 1 for at least 5 hours, more preferably 7.5 hours, still more preferably 10 hours or longer.

A large dose of cysteamine precursor (e.g. 2-10 grams) may be necessary to effectively treat some cysteamine-sensitive diseases, and/or to achieve adequate blood levels in large adult subjects. Since the amount of any active agent that can be contained in standard dosage forms (e.g. tablets, capsules) is limited by the ability of patients to swallow large compositions, and further since the administration of multiple tablets or capsules can be inconvenient or unpleasant (or impossible for patients with dysphagia), alternative dosage forms that do not constrain the amount of active agent in a unit dosage form are useful. Powders, granules and liquids are examples of non-size limited dosage forms, which can nevertheless be delivered in unit dosage amounts by suitable packaging, e.g. in a sachet or vial. In some embodiments of the present invention a floating gastroretentive composition of the invention is administered in liquid form. In a further embodiment the liquid composition includes alginate. In other embodiments active pharmaceutical ingredients are delivered in the form of a powder or granules that can be sprinkled on food.

One type of liquid gastroretentive floating drug delivery system utilizes alginate as an excipient. Alginic acid is a linear block polysaccharide copolymer made of beta-D-mannuronic acid and alpha-L-guluronic acid residues connected by 1,4 glycosidic linkages. It is used for a wide variety of purposes in pharmaceutical compositions, including as a sustained release polymer (see Murata et al., Eur J Pharm Biopharm 50:221 (2000)). Gaviscon is the brand name of a floating liquid alginate formulation that contains an antacid. It has been used to treat gastroesophageal reflux for decades, so the safety of chronic alginate ingestion is well established. Floating formulations of alginate with small molecule drugs have been described (see Katayama et al., Biol Pharm Bull. 22:55 (1999); and: Itoh et al., Drug Dev Ind Pharm. 36:449 (2010)). Floating formulations that form a layer on the surface of the stomach contents are sometimes referred to as raft-forming formulations. Raft-forming floating/gelling sustained release compositions have been described by Prajapati et al., J Control Release 168:151 (2013); and by Nagarwal et al., Curr Drug Deliv. 5:282 (2008).

U.S. Pat. No. 4,717,713, herein incorporated by reference, discloses liquid (drinkable) formulations that, upon contact with gastric contents, form a semi-solid gel-like matrix in the stomach, thereby effecting controlled release of a drug from the gelatinous matrix. Gel-forming vehicles are disclosed, including xanthan gum, sodium alginate, complex coacervate pairs such as gelatin or other polymers and carrageenan, and thermal gelling methylcellulose, all or a subset of which can be combined in various ratios to influence the dissolution and/or diffusion rate of suspended pharmaceutically active agent(s). Other excipients used include carbonate compounds such as calcium carbonate, effective as both a promoter of gelling and as a gas-generating agent to float the gel. Xyloglucans and gellan gums may also be used as gelling agents, or in combinations of gelling agents.

Liquid (drinkable) floating formulations may include microparticles, which may be provided as a liquid suspension (either a concentrate or ready for use) or as a powder which can be added to a liquid (e.g. water, juice or other beverage). Floating gastroretentive compositions may also be delivered in the form of powders to be sprinkled over, or otherwise mixed with, food.

Floating gastroretentive formulations may include mucoadhesive polymers or other mucoadhesive ingredients (see U.S. Pat. Nos. 6,207,197 and 8,778,396, incorporated herein by reference), and may utilize polymers such as polyethylene oxide, polyvinyl alcohol, sodium alginate, ethylcellulose, poly(lactic) co-glycolic acids (PLGA), polylactic acids, polymethacrylates, polycaprolactones, polyesters, polyacrylic acids and polyamides.

Swelling and Expanding Gastroretentive Compositions

Swelling and expansion is a gastric retention mechanism wherein, upon contact with gastric fluid the composition swells to an extent that prevents its exit from the stomach through the pylorus. As a result, the composition is retained in the stomach for a prolonged period of time, for example until the surface of the composition is eroded to reduce its diameter to less than the diameter of the pylorus, or until food is substantially emptied from the stomach, at which time strong muscular contractions (sometimes called the “housekeeper wave”) sweep across the stomach, clearing its contents. The composition is excluded from passing through the pyloric sphincter as it exceeds a diameter of approximately 14-16 mm in the swollen or expanded state. Preferably the composition exceeds a diameter of 16-18 mm. Swelling may be combined with floating, which keeps the formulation away from the pylorus, particularly in the fed state.

The concept of a formulation which swells upon contact with gastric fluid and consequently is retained in the stomach is known since the 1960s. U.S. Pat. No. 3,574,820 discloses tablets which swell in contact with gastric fluid to such a size that they cannot pass the pylorus and therefore are retained in the stomach. Similarly, U.S. Pat. No. 5,007,790 describes tablets or capsules composed of hydrophilic, water-swellable, cross-linked polymers that quickly swell to promote gastric retention, while allowing slow dissolution of drug molecules mixed with the polymers.

U.S. Patent Publication No. 20030104053, incorporated herein by reference, discloses unit dosage form tablets for the delivery of pharmaceuticals wherein the active component is dispersed in a solid unitary matrix that is formed of a combination of poly (ethylene oxide) and hydroxypropyl methylcellulose. This combination is said to offer unique benefits in terms of release rate control and reproducibility while allowing both swelling of the tablet to effect gastric retention and gradual disintegration of the tablet to clear the tablet from the gastrointestinal tract after release of the drug has occurred. U.S. Pat. No. 6,340,475, also assigned to DepoMed, herein incorporated by reference, highlights unit oral dosage forms of active components developed by incorporating them into polymeric matrices comprised of hydrophilic polymers that swell upon imbibing water to a size that is large enough to promote retention of the dosage form in the stomach during the fed mode. The polymeric matrix is formed of a polymer selected from the group consisting of poly (ethylene oxide), cellulose, crosslinked polyacrylic acids, xanthan gum and alkyl-substituted celluloses like hydroxymethyl-cellulose, hydroxyethyl-cellulose, hydroxypropyl-cellulose, hydroxypropylmethyl-cellulose, carboxymethyl-cellulose and microcrystalline cellulose.

Further, swelling gastroretentive systems based on gums have also been developed by DepoMed researchers. U.S. Pat. No. 6,635,280, incorporated herein by reference, discloses controlled release oral dosage forms for highly water soluble drugs comprising one or more polymers forming a solid polymeric matrix which swells upon imbibition of water to a size that is large enough to promote retention of the dosage form in the stomach during the fed mode. A polymeric matrix may be formed of a polymer selected from the following: poly(ethylene oxide), cellulose, alkyl-substituted celluloses, crosslinked polyacrylic acids, and xanthan gum. U.S. Pat. No. 6,488,962, incorporated herein by reference, discloses optimal tablet shapes that prevent passage through the pylorus while remaining convenient to swallow. The tablets are made using water swellable polymers including cellulose polymers and their derivatives, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, maltodextrins, poly (2-ethyl-2-oxazoline), poly(ethyleneimine), polyurethane hydrogels, crosslinked polyacrylic acids and their derivatives, as well as copolymers of the above listed polymers, including block copolymers and graft polymers.

U.S. Pat. No. 6,723,340, incorporated herein by reference, discloses optimal polymer mixtures for making swelling gastroretentive compositions. The mixtures provide optimal control of swelling and drug release parameters as well as control of dissolution/erosion parameters, so as to ensure passage of the composition into the small intestine upon substantially complete drug release. Preferred polymer mixtures include combinations of poly(ethylene oxide) and hydroxypropyl methylcellulose. Preferred molecular weight ranges and viscosity ranges are provided for the polymer mixtures.

The methods described in the foregoing patent publications have been used to formulate four U.S. FDA approved swelling gastroretentive formulations described in multiple publications (e.g. reviewed in: Berner et al., Expert Opin Drug Deliv. 3:541 (2006)).

U.S. Patent Publication No. 20080220060, incorporated herein by reference, discloses gastroretentive formulations comprising an active substance granulated with a mixture of a weak gelling agent, a strong gelling agent and a gas generating agent. Herein the strong gelling agent is selected from the group consisting of methyl cellulose, hydroxypropyl methyl cellulose, hydroxypropyl cellulose with the exclusion of low-substituted hydroxypropyl cellulose, hydroxyethyl cellulose, ethyl cellulose, sodium carboxymethyl cellulose, xanthan gum, guar gum, carrageenan gum, locust bean gum, sodium alginate, agar-agar, gelatin, modified starches, co-polymers of carboxyvinyl polymers, co-polymer of acrylates, co-polymers of oxyethylene and oxypropylene and mixtures thereof. The patent also describes manufacturing methods. U.S. Pat. No. 7,674,480 discloses swelling gastroretentive formulation methods that provide for very rapid swelling using mixtures including a superdisintegrant, tannic acid and one or more hydrogels. U.S. Patent Publication No. 20040219186, incorporated herein by reference, provides expandable gastric retention device comprising a gel formed from a polysaccharide, based on xanthan gum or locust bean gum or a combination thereof. U.S. Patent Publication No. 20060177497, incorporated herein by reference, discloses gellan gum based oral controlled release dosage forms as a platform technology for gastric retention. The dosage form further comprises hydrophilic polymers such as guar gum, hydroxypropyl methylcellulose, carboxymethyl cellulose sodium salt, xanthan gum.

U.S. Pat. No. 6,660,300 discloses a biphasic swelling gastroretentive formulation technology, suitable for delivering water soluble drugs, in which swelling and drug release are accomplished by separate compartments of a composition: an inner solid particulate phase contains the drug and one or more hydrophilic polymers, one or more hydrophobic polymers and/or one or more hydrophobic materials such as waxes, fatty alcohols and/or fatty acid esters. An outer solid continuous phase (in which granules of the drug-containing inner phase are embedded) is formed using one or more hydrophobic polymers and/or one or more hydrophobic materials such as waxes, fatty alcohols and/or fatty acid esters. Tablets and capsules are disclosed.

Other excipients useful In a swelling or expandable matrix formulation include (i) a water-swellable polymer matrix and (ii) hydrophilic polymers selected from the following: polyalkylene oxides, particularly poly(ethylene oxide), polyethylene glycol and poly(ethylene oxide)-poly(propylene oxide) copolymers; cellulosic polymers; acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate; maleic anhydride copolymers; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol), poly(N-vinyl lactams) such as poly(vinyl pyrrolidone), poly(N-vinyl caprolactam), and copolymers thereof polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol and polyoxyethylated glucose; polyoxazolines, including poly(methyloxazoline) and poly(ethyloxazoline); polyvinylamines; polyvinylacetates, including polyvinylacetate per se as well as ethylene-vinyl acetate copolymers, polyvinyl acetate phthalate, and the like, polyimines, such as polyethyleneimine; starch and starch-based polymers; polyurethane hydrogels; chitosan; polysaccharide gums; zein; and shellac, ammoniated shellac, shellac-acetyl alcohol, and shellac n-butyl stearate. The gastroretentive formulation may also include any combination of a floating formulation, mucoadhesive formulation, expandable matrix formulation, modified shape formulation and/or a magnetic formulation. In some embodiments the pharmaceutical composition of the present invention is a gastroretentive composition which is retained in the stomach as a result of swelling to a size that inhibits passage through the pylorus. In further embodiments the gastroretentive composition is retained in the stomach by both swelling and floating mechanisms.

Unfolding, Shape-Changing Gastroretentive Formulations

Pharmaceutical compositions that unfold, decompress or otherwise change size and/or shape upon contact with liquid gastric contents have also been described and are suitable delivery vehicles for the compounds and formulations of the invention. Such compositions employ a similar principal to swelling/expanding gastroretentive formulations in that they change shape in the stomach to a size and/or geometry that does not easily permit passage through the pylorus. Methods and materials for making unfolding, uncoiling or other shape-changing gastroretentive compositions are known in the art. For example U.S. Pat. No. 3,844,285 describes a variety of such devices intended for veterinary use in ruminants, however the basic principles also apply to human gastroretentive formulations. U.S. Pat. No. 4,207,890 describes a controlled release drug delivery system consisting of a “collapsed, expandable, imperforate polymer envelope containing within it an effective expanding amount of an expanding agent, agent” which swells and unfolds on contact with gastric juice, and is consequently retained in the stomach in the expanded state. The composition is administered inside a capsule in collapsed form. Unfolding and shape changing gastroretentive compositions have been reviewed (e.g. Klausner et al., Journal of Controlled Release 90:143 (2003)).

An exemplary unfolding gastroretentive technology called the “Accordion Pill” is being developed by Intec Pharma (Jerusalem, Israel). Multi-layer planar structures of various shapes (in which at least one layer contains a drug) are folded into an accordion or staircase-like shape and packaged inside a capsule, as described in: Kagan, L. Journal of Controlled Release 113:208 (2006). Additional features of the Accordion Pill and related technologies are disclosed in U.S. Pat. No. 6,685,962, herein incorporated by reference, including pharmaceutical excipients preferably used in its construction. The capsule dissolves upon contact with stomach contents, releasing a folded composition which rapidly unfolds and is thereafter retained in the stomach for up to 12 hours when administered with a regular meal.

Other gastroretentive technologies include superporous hydrogels and Ion exchange resin systems. Superporous hydrogels swell rapidly (within a minute of contacting liquid) due to rapid water uptake via numerous interconnected pores. Compositions may swell up to 100 times or more their original size, yet retain sufficient mechanical strength to withstand the forces of gastric contraction due to co-formulation with hydrophilic polymers such as croscarmellose sodium (e.g. brand name: Ac-Di-Sol). Ion exchange resin beads can be loaded with negatively charged drugs and made to float using gas generating agents (e.g. bicarbonate, which reacts with chloride ion in the gastric fluid to generate carbon dioxide gas). The beads are encapsulated in a semi-permeable membrane which traps the gas, resulting in long-term flotation of the beads.

Gastroretentive formulations may also include any combination of a mucoadhesive, floating, raft-forming, swelling, unfolding/shape changing, superporous hydrogel or ion exchange resin formulation. Such combinations are known to those skilled in art. For example U.S. Pat. No. 8,778,396 (“Multi-unit gastroretentive pharmaceutical dosage form comprising microparticles”), herein incorporated by reference in its entirety, describes a combined mucoadhesive floating gastroretentive formulation consisting of microparticles.

The compositions of the present invention may include, but are not limited to, hydrophilic polymers having swelling and/or mucoadhesive properties to further promote gastroretention. Hydrophilic polymers having swelling and/or mucoadhesive properties suitable for incorporation in the compositions of present invention include, but are not limited to, polyalkylene oxides; cellulosic polymers; acrylic acid and methacrylic acid polymers, and esters thereof, maleic anhydride polymers; polymaleic acid; poly(acrylamides); poly(olefinic alcohol)s; poly(N-vinyl lactams); polyols; polyoxyethylated saccharides; polyoxazolines; polyvinylamines; polyvinylacetates; polyimines; starch and starch-based polymers; polyurethane hydrogels; chitosan; polysaccharide gums; zein; shellac-based polymers; polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, maltodextrin, pregelatinized starch and polyvinyl alcohol, copolymers and mixtures thereof.

Release of active ingredients from a composition may be achieved through use of suitable retardants that include excipients well known in the pharmaceutical art for their release retarding properties. Examples of such release retardants include, but are not limited to, polymeric release retardants, non-polymeric release retardants or any combinations thereof.

Polymeric release retardants employed for the purpose of the present invention include, but are not limited to, cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; or any combinations thereof. Cellulose derivatives include, but are not limited to, ethyl cellulose, methylcellulose, hydroxypropylmethylcellulose (H PMC), hydroxypropyl cellulose (HPC), hydroxyethyl cellulose, hydroxymethyl cellulose, hydroxyethyl methyl cellulose, carboxymethyl cellulose (CMC), or combinations thereof. Polyhydric alcohols include, but are not limited to, polyethylene glycol (PEG) or polypropylene glycol; or any combinations thereof. Saccharides, gums and their derivatives include, but are not limited to, dextrin, polydextrin, dextran, pectin and pectin derivatives, alginic acid, sodium alginate, starch, hydroxypropyl starch, guar gum, locust bean gum, xanthan gum, karaya gum, tragacanth, carrageenan, acacia gum, arabic gum, fenugreek fibers or gellan gum or the like; or any combinations thereof. Vinyl derivatives, polymers, copolymers or mixtures thereof include, but are not limited to, polyvinyl acetate, polyvinyl alcohol, mixtures of polyvinyl acetate (8 parts w/w) and polyvinylpyrrolidone (2 parts w/w) (Kollidon SR), copolymers of vinyl pyrrolidone, vinyl acetate copolymers, polyvinylpyrrolidone (PVP); or combinations thereof. Polyalkylene oxides or copolymers thereof include, but are not limited to, polyethylene oxide, polypropylene oxide, poly (oxyethylene)-poly (oxypropylene) block copolymers (poloxamers) or combinations thereof. Maleic acid copolymers include, but are not limited to, vinylacetate maleic acid anhydride copolymer, butyl acrylate styrene maleic acid anhydride copolymer or the like or any combinations thereof. Acrylic acid polymers and acrylic acid derivatives include, but are not limited to, carbomers, methacrylic acids, polymethacrylic acids, polyacrylates, polymethacrylates or the like or combinations thereof. Polymethacrylates, include, but are not limited to, a) copolymer formed from monomers selected from methacrylic acid, methacrylic acid esters, acrylic acid and acrylic acid esters c) copolymer formed from monomers selected from ethyl acrylate, methyl methacrylate and trimethylammonioethyl methacrylate chloride, or the like or any combinations thereof. Non-polymeric release retardants employed for the purpose of the present invention include, but are not limited to, fats, oils, waxes, fatty acids, fatty acid esters, long chain monohydric alcohols and their esters or combinations thereof. In an embodiment, non-polymeric release retardants employed in the present invention, include, but are not limited to, Cutina (hydrogenated castor oil), Hydrobase (hydrogenated soybean oil), Castorwax (hydrogenated castor oil), Croduret (hydrogenated castor oil), Carbowax, Compritol (glyceryl behenate), Sterotex (hydrogenated cottonseed oil), Lubritab (hydrogenated cottonseed oil), Apifil (wax yellow), Akofine (hydrogenated cottonseed oil), Softtisan (hydrogenated palm oil), Hydrocote (hydrogenated soybean oil), Corona (lanolin), Gelucire (macrogolglycerides lauriques), Precirol (glyceryl palmitostearate), Emulcire (cetyl alcohol). Plurol diisostearique (polyglyceryl diisostearate), and Geleol (glyceryl stearate), and mixtures thereof.

The gastroretentive compositions of the present invention may be in a form such as, but not limited to, a monolithic or multi-layered dosage form or in-lay system. In one embodiment of the present invention the gastroretentive compositions are in the form of a bilayered or trilayered solid dosage form. In an illustrative embodiment, a solid pharmaceutical composition in the form of an expanding bilayered system for oral administration is adapted to deliver an active pharmaceutical component from a first layer immediately upon reaching the gastrointestinal tract, and to deliver a further pharmaceutical agent which may be same or different from a second layer, in a modified manner over a specific time period. The second layer may be formulated to expand in the composition, thereby prolonging retention of the composition in the stomach.

In a further illustrative embodiment a solid pharmaceutical composition for oral administration contains two layers: one comprising an active component along with a suitable release retardant and the other layer comprising swellable agent in combination with other excipients. In another embodiment of the present invention, a solid pharmaceutical composition for oral administration contains an in-lay system which is a specialized dosage form comprising a first tablet containing active component(s) which is placed inside a second tablet comprising excipients that ensure gastric retention. In this system the active component containing tablet is small and is covered on all sides except at least one side with a blend of excipient comprising swellable polymers or a flotation system, or both, that ensures gastric retention. In yet another embodiment of the present invention, the dosage form may be optionally coated.

Surface coatings may be employed for organoleptic purposes (particularly with thiols or disulfides that have an odor, or an unpleasant taste), for drug labeling purposes (e.g. a color coding system for dosage forms), for aesthetic purposes, for dimensionally stabilizing the compressed dosage form, or for retarding drug release. The surface coating may be any conventional coating which is suitable for enteral use. The coating may be carried out using any conventional technique employing conventional ingredients. A surface coating can for example be obtained using a quick-dissolving film using conventional polymers such as, but not limited to, hydroxypropyl methyl cellulose, hydroxypropyl cellulose, carboxymethyl cellulose, polyvinyl alcohol, poly methacrylates or the like. Coating excipients and methods for using them are well known in the art. See for example: McGinity, James W. and Linda A. Felton, Aqueous Polymeric Coatings for Pharmaceutical Dosage Forms, Third Edition, Informa Healthcare, 2008.

Further, in another embodiment of the present invention, the compositions are in the form of multiparticulates including, but not limited to, pellets, microspheres, microcapsules, microbeads, microparticles or nanoparticles having prolonged transit in the intestine to effectively deliver active agents that require longer retention times in the intestinal tract. Multiparticulate systems may be (i) bioadhesive or mucoadhesive, thereby delaying gastrointestinal transit, or (ii) may float on top of the gastric contents, optionally forming a gel-like layer, or (iii) may be coated with a pH sensitive outer layer or layers that dissolve in the mildly acidic environment of the small intestine, or in the neutral to slightly basic environment of the ileum (typically the gut segment with the highest pH), or (iv) may be formed using a drug containing polymer that is not digestible by human enzymes but is digestible by enzymes produced by enteric bacteria, leading to drug release in the distal ileum and colon. In an embodiment, the compositions of the present invention, in the form of multiparticulates, are gastroretentive. Such multiparticulate systems may be prepared by methods including, but not limited to, pelletization, granulation, spray drying, spray congealing and the like.

A suitable polymeric release controlling agent may be employed in the compositions of the present invention. In one embodiment, the polymeric release controlling agent is pH independent or pH dependent or any combination thereof. In another embodiment, the polymeric release controlling agent employed in the compositions of the present invention may be swelling or non-swelling. In a further embodiment, polymeric release controlling agents that may be employed in the compositions of the present invention include, but are not limited to, cellulose derivatives, saccharides or polysaccharides, poly(oxyethylene)-poly(oxypropylene) block copolymers (poloxamers), vinyl derivatives or polymers or copolymers thereof, polyalkylene oxides and derivatives thereof, maleic copolymers, acrylic acid derivatives or the like or any combinations thereof.

Controlled release compositions for oral use may be constructed to release the active drug by controlling the dissolution and/or the diffusion of the active drug substance. Any of a number of strategies can be pursued in order to obtain controlled release and thereby optimize the plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, liquids, suspensions, emulsions, microcapsules, microspheres, nanoparticles, powders and granules. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

Alternatively, certain cysteamine precursors or enhancers of in vivo cysteamine generation or absorption may be formulated and administered as medical foods. Medical foods are regulated by the US FDA as foods, not drugs. Methods for formulating medical foods are known in the art. See, for example, U.S. Patent Publication No. 20100261791, for descriptions of methods for preparing and administering active compounds in foods or beverages. Nutracia, a medical food company based in The Netherlands, has over 250 patent applications and patents describing methods for combining pharmacologically active agents with foods or drinks.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract. Alternatively, leaky enteric coatings may be used to provide a release profile intermediate between immediate release and delayed release formulations. For example U.S. patent application 20080020041 A1 discloses pharmaceutical formulations coated with an enteric material that releases at least a portion of an active ingredient upon contacting gastric fluid, with the remainder released upon contacting intestinal fluid.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

For controlled release formulations, the active component of the composition may be targeted for release in the small intestine. The formulation may contain an enteric coating such that the composition is resistant to the low pH environment found in the stomach, but sensitive to the higher pH environment of the small intestine. To control the release of the active component in the small intestine, a multiparticulate formulation may be employed to prevent simultaneous release of the active component. A multiparticulate composition may include a plurality of individual enteric coated cores that include a hydrophobic phase containing a cysteamine precursor, or a salt thereof, dispersed in a microcrystalline cellulose-based gel and a hydrophilic phase containing a hydrogel. The microcrystalline cellulose (MCC) functions as a release controlling polymer for the cysteamine precursor, or a salt thereof, preventing dose dumping and stabilizing the cysteamine precursor, or a salt thereof, while the cores are being dissolved or eroded in the intestine. Two or more multiparticulate compositions that differ with respect to excipients in the core or the coating layer may be combined in one pharmaceutical composition (e.g. a capsule, powder or liquid) so as to release active ingredients (e.g. cysteamine precursors) over a longer time period. Alternatively the same effect can be achieved by using different concentrations of excipients in two or more batches of microparticles and then combining the microparticles from different batches in a chosen ratio (e.g. 1:1) so as to effect a targeted drug release profile.

The composition may include a plurality of individual enteric coated cores containing about 15% w/w to about 70% w/w cysteamine precursor, or a salt thereof, about 25% w/w to about 75% w/w microcrystalline cellulose, and about 2% w/w to about 15% w/w methylcellulose, wherein the % w/w is the w/w of the enteric coated cores.

In some cases, including a continuous proteinaceous subcoating layer covering the individual cores and separating the individual cores from their respective enteric coatings may be advantageous because the proteinaceous subcoating layer further enhances the stability of the cysteamine precursor, or a salt thereof. The continuous proteinaceous subcoating is adapted to prevent the cysteamine precursor, or a salt thereof, from mixing with the enteric coating. Some preferred proteinaceous subcoatings have the following attributes: the subcoating may comprise a gelatin film adhered to the core and/or the subcoating may comprise a dried proteinaceous gel.

In a particular embodiment, the enteric coated cores release no more than about 20% of the cysteamine precursor, or a salt thereof, within about two hours of being placed in a 0.1 N HCl solution and, subsequently, no less than about 85% of the cysteamine precursor, or a salt thereof, within about eight hours of being placed in a substantially neutral pH environment.

Preferably, the enteric coated cores are spheroidal and not more than 3 mm in diameter.

To prevent adherence of separately administered compositions in the stomach, compositions of the invention may be coated with an anti-adhering agent. Anti-adherents may also be used to prevent microparticles from sticking to each other. For example, compositions may be coated with a thin outermost layer of microcrystalline cellulose powder. Alternatively, adherence can be prevented by coating with a polymer that is insoluble in gastric juice but permeable and swellable. For example a 30% polyacrylate dispersion (e.g. Eudragit NE30D, Evonik Industries) has been shown to prevent adherence of floating minitablets in the stomach (see Rouge et al., European Journal of Pharmaceutics and Biopharmaceutics 43:165 (1997)).

Commercial forms of the listed excipients used in enteric coatings include, for example, various brands of polymethacrylates (a chemically heterogeneous group of compounds that includes amino methacrylate copolymer, ammonio methacrylate copolymer, ethyl acrylate copolymer dispersion, methyl methacrylate copolymer dispersion, methacrylic acid copolymer and methacrylic acid copolymer dispersion) which are sold as product lines by companies including, without limitation, Ashland, BASF Fine Chemicals (Kollicoat product line), ColorCon (Acryl-EZE product line), Eastman Chemical (Eastacryl product line) and Evonik Industries (Eudragit product line).

Formulations for Ileal and Colonic Drug Release

In some embodiments, ileum and/or colon-targeted formulations can be used to deliver cysteamine precursors to the distal ileum and colon. (The term “colon targeted” is used herein to refer to both ileum-targeted and colon-targeted formulations; any composition that starts to release drug in the ileum is likely to also release drug in the colon, and some drug released in the ileum is likely to reach the colon.) Drug delivery advantages of colon-targeted compositions include prolonged contact with the large intestinal epithelium and the presence of colonic bacteria that can be exploited for site specific delivery.

From a pharmacokinetic perspective colonic absorption of cysteamine is desirable because, due to its extremely short half life, cysteamine must be continuously produced in the gastrointestinal tract (and absorbed) to maintain blood levels in the therapeutic range. An ingested pharmaceutical composition (if not a gastroretentive composition) may arrive in the colon three to five hours after ingestion (on average, in most subjects) if ingested in the fasted condition, or six to 10 hours (on average, in most subjects) after ingestion with food. The only way to sustain blood cysteamine levels in the therapeutic range after the dosage form reaches the colon is to ensure cysteamine is generated and absorbed in the colon. Some cysteamine precursors released in the small intestine may pass into the colon intact and be degraded to cysteamine in the colon. However, to provide robust cysteamine generation in the colon cysteamine precursors should be formulated for release in the colon (or ileum), where they can be degraded to cysteamine and absorbed. Colon-targeted compositions are not intended to be used alone as therapy for cysteamine-sensitive diseases, but rather to complement formulations directed to other areas of the gastrointestinal tract.

Two approaches to colon-targeted delivery have been developed extensively and are described below.

The first approach involves exploitation of enzymes produced in the colon by enteric bacteria. Enteric bacteria can digest a variety of polymers that are indigestible by human enzymes present in saliva, gastric juice, intestinal fluid or pancreatic juice. Pharmaceutical compositions containing such polymers cannot be digested—and therefore active ingredients admixed with the polymers cannot escape—until they encounter enzymes produced by enteric bacteria in the distal ileum (where the density of bacteria starts to increase) or the colon (where there may be 1,000,000,000,000 bacteria per milliliter of colon contents).

A cysteamine precursor and/or other active ingredient (e.g. an enhancer of in vivo cysteamine generation or absorption) can be mixed with a polymer that retards drug release and is only digestible (in the human gastrointestinal tract) by enzymes produced by enteric bacteria. Polymers used for colon-targeted drug delivery based on selective degradation by enteric bacteria include dextran hydrogels (Hovgaard, L., and H. Brondsted, J. Controlled Rel. 36:159 (1995)), crosslinked chondroitin (Rubinstein et al., Pharm. Res. 9:276 (1992)), and hydrogels containing azoaromatic moieties (Brondsted, H. and J. Kopoecek, Pharm Res. 9:1540 (1992); and Yeh et al., J. Controlled Rel. 36:109 (1995)).

Covalent linkage of a drug with a carrier to form a precursor that is stable in the stomach and small intestine and releases the drug in the large intestine upon enzymatic cleavage by the intestinal microflora; examples of these precursors include azo-conjugates, cyclodextrin-conjugates, glycoside-conjugates, glucuronate conjugates, dextran-conjugates, polypeptide and polymeric conjugates. The basic principle is that the covalent bond linking drug to carrier must be indigestible by human enzymes but digestible by enteric bacterial enzymes.

The second approach involves exploitation of high pH in the ileum relative to other parts of the gastrointestinal tract. In healthy subjects the pH in the gastrointestinal tract increases from the duodenum (approximately pH 5.5 to 6.6 from the proximal to the distal duodenum) to the terminal ileum (approximately pH 7-7.5), then decreases in the cecum (around pH 6.4), and then increases again from the right to the left side of the colon with a final value of about pH 7.

Compositions may be coated with a pH-sensitive polymer that dissolves only at neutral to mildly alkaline pH (e.g. above pH 6.5, above pH 6.8 or above pH 7). Beneath the pH sensitive coating is a sustained release formulation from which drug is slowly released by diffusion, erosion or a combination. This approach is described in U.S. Pat. No. 5,900,252, incorporated herein by reference.

The enteric bacterial and pH based colon targeting methods can be combined. See, for example: Naeem et al., Colloids Surf B Biointerfaces S0927 (2014). The study describes coated nanoparticles formed using bacteria-digestible polymers. Another technology that combines pH and bacterial enzyme digestion to deliver drug-containing liquid-filled capsules to the colon is described in U.S. Patent Publication No. 20070243253, which discloses formulations that utilize polymers including starch, amylose, amylopectin, chitosan, chondroitin sulfate, cyclodextrin, dextran, pullulan, carrageenan, scleroglucan, chitin, curdulan and levan, together with pH sensitive coatings that dissolve above about pH 5 or higher.

Other approaches to colon-targeted drug delivery employ: (i) time release systems where once a multicoated formulation passes the stomach the outer coat starts to dissolve and, based on the thickness and compostions of the coatings, drug is released after a lag time of 3-5 hrs, which is about the transit time of the small intestine; (ii) redox-sensitive polymers where a combination of azo- and disulfide polymers, provide drug release in response to the low redox potential of the colon; (iii) bioadhesive polymers which selectively adhere to the colonic mucous, slowing transit of the dosage form to allow drug release the drug; and/or (iv) osmotic controlled drug delivery where drug is released through a semi-permeable membrane due to osmotic pressure.

The book “Oral Colon-Specific Drug Delivery” by David R. Friend (CRC Press, 1992) provides and overview of older colon-targeting methods (many of which are still useful), such as dextran-based delivery systems, glycoside/glycosidase-based delivery, azo-bond prodrugs, hydroxypropyl methacrylamide copolymers and other matrices for colon delivery. Colon-targeted drug delivery has been reviewed more recently by, for example: Bansal et al., Polim Med. 44:109 (2014). Recent approaches include use of novel polymers digestible only by enzymes produced by enteric bacteria, including natural polymers found in a variety of plants, as well as microbeads, nanoparticles and other microparticles.

### Methods of Treatment

The present invention features methods useful for treating betacoronavirus infections. Treatment entails oral administration of cysteamine precursors, convertible to cysteamine in the gastrointestinal tract. An important class of cysteamine precursors are mixed disulfides which, upon reduction in vivo, provide two thiols. Both thiols may be convertible to cysteamine in vivo, or just one. Cysteamine precursors in which both thiols are convertible to cysteamine are a preferred class of therapeutic agents for diseases including cystinosis, cystic fibrosis, malaria, and viral and bacterial infections. Non-limiting examples of such mixed disulfides include cysteamine-pantetheine and cysteamine-4-phosphopantetheine.

Dosing Regimens

The present methods for modulating plasma cysteamine levels in the treatment of betacoronavirus infections are carried out by administering one or more compositions containing one or more cysteamine precursors and optionally one or more enhancers of in vivo cysteamine generation and/or absorption for a time and in an amount sufficient to result in elevated plasma levels of cysteamine adequate to provide an effective treatment of a betacoronavirus infection. For example, while both gastroretentive and non-gastroretentive sustained release formulations can, by themselves, provide cysteamine precursor release over 3, 5, 8 or more hours, it may be desirable, in order to achieve more steady blood levels of cysteamine in the therapeutic concentration range for longer time periods, to co-administer either of those formulation types with one or more other compositions, such as an immediate release, delayed release or colon-targeted composition. Compostions that contain two types of formulation, referred to as mixed formulations, may also be administered.

The amount and frequency of administration of the compositions can vary depending on, for example, what is being administered (e.g. which cysteamine precursors, which enhancers, which types of formulation), the disease, the state of the patient, and the manner of administration. In therapeutic applications, compositions can be administered to a patient suffering from elevated WBC cysteine levels (e.g., cystinosis) in an amount sufficient to decrease or least partially decrease the WBC cysteine levels, preferably below recommended levels. The dosage is likely to depend on such variables as the type and extent of progression of the disease, the age, weight and general condition of the particular patient, the the route of administration, and the judgment of the attending clinician. Effective doses can be estimated from dose-response curves derived from in vitro or animal model test system. An effective dose is a dose that produces a desirable clinical outcome.

The amount of a cysteamine precursor, or salt thereof per dose can vary. The upper end of the dose range for cysteamine bitartrate is 1.95 grams per square meter of body surface area per day (only counting the weight of the cysteamine), which amounts to about 3.7 grams/day of cysteamine base for an average adult. However, that amount of cysteamine is associated with significant side effects and in some cases discontinuation of therapy.

The molecular weight of cysteamine precursors varies widely, as does the fraction convertible to cysteamine in vivo. Several examples may serve to illustrate the variation. The molecular weight of cysteamine base is 77.15 g/mol. The molecular weight of the thiol pantetheine is 278.37 g/mol. Therefore a cysteamine-pantetheine disulfide has a molecular weight of approximately 353.52 (adjusting for two protons lost in the oxidation reaction) and is convertible in vivo to two cysteamines which together weigh 154.3. Thus about 43.6% of a cysteamine-pantetheine disulfide is convertible to cysteamine. Assuming 100% conversion of the cysteamine-pantetheine disulfide to cysteamine in vivo, and further assuming equivalent bioavailability, a maximum dose of cysteamine-pantetheine disulfide is in the range of 8.5 grams/day for a 70 kg adult, or about 0.12 grams/kg/day. The bioavailability of cysteamine precursors, when dosed to match the in vivo cysteamine generating and absorbing capacity of a patient, is expected to be moderately higher than that of cysteamine salts. In vivo conversion of cysteamine precursors to cysteamine is unlikely to be 100%, but very high rates of conversion can be achieved by calibration of dosing regimens to pharmacokinetic parameters, and by co-administration of appropriate enhancers of cysteamine precursor breakdown and absorption.

The disulfide pantethine has a molecular weight of 554.723 g/mol and, upon reduction and pantetheinase cleavage yields two molecules of cysteamine (i.e. 27.8% of pantethine will become cysteamine). Thus, making the same assumptions as above, a maximum dose of pantethine is in the range of 13 grams/day for a 70 kg adult, or about 0.19 grams/kg/day.

For a large cysteamine precursor like coenzyme A (MW 767.535 g/mol), that only yields one molecule of cysteamine, the fraction of a dose convertible to cysteamine is only about 10%, and consequently the maximum dose of coenzyme A could be up to 37 grams/day for a 70 kg adult, or about 0.5 grams/kg/day. For that reason coenzyme A is not preferred as a sole treatment for diseases that require high blood levels of cysteamine for good therapeutic effect, but may be combined with other cysteamine precursors that more efficiently deliver cysteamine.

The low end of the useful range of cysteamine precursor doses is not determined by side effects and tolerability limits, but entirely by efficacy, which may vary considerably from one disease to another. For example, because first pass metabolism by the liver (which clears about 40% of absorbed cysteamine from the blood) does not affect cysteamine delivery to the liver the range of effective doses for liver diseases is lower than for other diseases.

For example, a subject can receive from about 0.01 g/kg to about 0.5 g/kg of a cysteamine precursor. Generally, the cysteamine and pantetheine compound is administered in an amount such that the peak plasma concentration ranges from 1 μM-45 μM. Exemplary dosage amounts can fall between about 0.01 to about 0.2 g/kg; about 0.05 to about 0.2 g/kg; about 0.1 to about 0.2 g/kg; about 0.15 to about 0.2 g/kg; about 0.05 g/kg to about 0.25 g/kg; about 0.1 g/kg to about 0.25 g/kg; about 0.15 g/kg to about 0.25 g/kg; about 0.1 g/kg to about 0.50 g/kg; about 0.2 to about 0.5 g/kg; about 0.3 to about 0.5 g/kg; or about 0.35 to about 0.5 g/kg. Exemplary dosages can be about 0.005 g/kg, about 0.01 g/kg, about 0.015 g/kg, about 0.02 g/kg, about 0.03 g/kg, about 0.05 g/kg, about 0.1 g/kg, about 0.15 g/kg, about 0.2 g/kg or about 0.5 g/kg. Exemplary peak plasma concentrations can range from 5-20 μM, 5-15 μM, 5-10 μM, 10-20 μM, 10-15 μM, or 15-20 μM. The peak plasma concentrations may be maintained for 2-14 hours, 4-14 hours, 6-14 hours, 6-12 hours, or 6-10 hours.

The frequency of treatment may also vary. The subject can be treated one or more times per day (e.g., once, twice, or thrice) or every so-many hours (e.g., about every 8, 12, or 24 hours). Preferably, the pharmaceutical composition is administered 1 or 2 times per 24 hours. The time course of treatment may be of varying duration, e.g., for two, three, four, five, six, seven, eight, nine, ten, or more days, two weeks, or 1 month. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

The invention also features kits useful in carrying out the methods of the invention. A kit can contain all active and inactive ingredients in unit dosage form or the active ingredient and inactive ingredients in two or more separate containers, and can contain instructions for administering or using the pharmaceutical composition to treat a betacoronavirus infection.

In some embodiments, a kit contains a cysteamine precursor compound or a pharmaceutically acceptable salt thereof, or a pharmaceutical composition comprising the same, and instructions for administering the compound or the pharmaceutical composition to treat or prevent a betacoronavirus infection.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1. Effect of Cysteamine Precursor Compounds on SARS-CoV-2 Replication

Cysteamine precursor compounds can be assessed in a Syrian hamster infection model (Chan et al., “Simulation of the clinical and pathological manifestations of Coronavirus Disease 2019 (COVID-19) in golden Syrian hamster model: implications for disease pathogenesis and transmissibility,” Clinical Infectious Diseases, ciaa325, (2020).

The efficacy of cysteamine-pantetheine disulfide (compound 1) is assessed in the Syrian hamster model. Maximal clinical signs of rapid breathing, weight loss, histopathological changes from the initial exudative phase of diffuse alveolar damage with extensive apoptosis to the later proliferative phase of tissue repair, airway and intestinal involvement with virus nucleocapsid protein expression, high lung viral load, and spleen and lymphoid atrophy associated with marked cytokine activation are observed within the first week of virus challenge. The lung virus titer is between 10⁵-10⁷ TCID₅₀/g. Challenged index hamsters can consistently infected naïve contact hamsters housed within the same cage, resulting in similar pathology but not weight loss. All infected hamsters can recover and develop mean serum neutralizing antibody titer ≥1:427 fourteen days post-challenge. Immunoprophylaxis with early convalescent serum can achieve significant decrease in lung viral load but not in lung pathology.

Three groups of 3 Syrian hamsters infected by SARS-CoV-2 per group receive cysteamine-pantetheine disulfide (compound 1) per os once a day for 7 days. The first group receives 60 mg/kg, second group 90 mg/kg and third group 120 mg/kg. Clinical status, biological parameters and viral load are characterized every day.

Animals infected with SARS-CoV-2 receiving treatment with compound 1 can experience a lower risk of sever COVID-19 outcomes, including a lower viral load, a more rapid recovery, and less severe symptoms of infection.

### Example 2. Safety, Tolerability and Pharmacokinetics (PK) and Pharmacodynamics (PD) of Cysteamine-Pantetheine Disulfide (Compound 1) in Patients with Coronavirus Disease 2019 (COVID-19)

A single-dose/multiple dose, open-labeled, non-randomized, two-period study of Cysteamine-Pantetheine Disulfide (compound 1) in up to patients (male and female) with COVID-19 under fasting or fed conditions is performed. Prior to treatment, patients undergo screening to determine study eligibility (Day-1).

On Day 0, patients receive 1 low dose (600 mg equivalent of cysteamine base) of compound 1 and PK and PD samples are collected according to the Schedule of Events.

On Day 3, patients receive a higher dose (1,200 mg equivalent of cysteamine base) of compound 1 and PK and PD samples are collected according to the Schedule of Events.

Based on the PK characteristics determined after single dose period, the first 6 patients receive a low dose of compound 1, once or twice a day, from Day 7 to Day 13, and the next 6 patients receive a high dose of compound 1, once or twice a day, from Day 7 to Day 13, without exceeding 1.95 g/m2/day equivalent of cysteamine base (˜4 g for a 70 kg adult).

The plasma levels of cysteamine, taurine, pantetheine and cysteamine-pantetheine (compound 1) are obtained from samples collected on Day 0 to Day 2, on Day 3 to Day 5 and on Day 7 to Day 15 according to the Schedule of Events.

The plasma concentration-time profiles of cysteamine, taurine, pantetheine and cysteamine-pantetheine (compound 1) are determined for each patient and the following plasma PK parameters estimated: Cmax, Tmax, t½ (elimination half-life), AUC0-t (area under the concentration-time curve from time 0 to time of the last quantifiable concentration), AUC0-inf (area under the concentration-time curve from time 0 extrapolated to infinity, AUC0-t/AUC0-inf (also known as AUCR), and Kel (terminal elimination rate constant).

Pharmacodynamic Evaluations: SARS-CoV-2 viral load will be obtained from samples collected from Day 0 to Day 15 according to the Schedule of Events.

At the end of the study, subjects infected with SARS-CoV-2 receiving treatment with compound 1 can experience a lower risk of sever COVID-19 outcomes, including a lower risk of pneumonitis, acute respiratory distress syndrome, respiratory failure, septic shock, organ failure, cytokine storm, and/or death.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of treating a betacoronavirus infection in a human subject, said method comprising administering to the subject a therapeutically effective amount of a cysteamine precursor compound or a pharmaceutically acceptable salt thereof.

2. A method of ameliorating one or more symptoms of a betacoronavirus infection in a human subject, said method comprising administering to the subject a therapeutically effective amount of a cysteamine precursor compound or a pharmaceutically acceptable salt thereof.

3. A method of inhibiting the progression of a betacoronavirus infection in a human subject, said method comprising administering to the subject a therapeutically effective amount of a cysteamine precursor compound or a pharmaceutically acceptable salt thereof.

4. A method of reducing the likelihood of betacoronavirus infection in a human subject at risk thereof, said method comprising administering to the subject a therapeutically effective amount of a cysteamine precursor compound or a pharmaceutically acceptable salt thereof.

5. The method of any one of claims 1-4, wherein the risk of pneumonia or pneumonitis in the subject is reduced.

6. The method of any one of claims 1-4, wherein the risk of hospitalization or the duration of hospitalization of the subject is reduced.

7. The method of any one of claims 1-4, wherein the risk of acute respiratory distress syndrome in the subject is reduced.

8. The method of any one of claims 1-4, wherein the risk of respiratory failure in the subject is reduced.

9. The method of any one of claims 1-4, wherein the risk of septic shock in the subject is reduced.

10. The method of any one of claims 1-4, wherein the risk of organ failure in the subject is reduced.

11. The method of any one of claims 1-4, wherein the risk of death in the subject is reduced.

12. The method of any one of claims 1-4, wherein the risk of cytokine storm in the subject is reduced.

13. The method of any one of claims 1-12, wherein the administering occurs between once per week to three times per day.

14. The method of any one of claims 1-12, wherein the administering occurs once per day.

15. The method of any one of claims 1-12, wherein the administering is twice per day.

16. The method of any one of claims 1-15, wherein the administering occurs over a treatment period.

17. The method of claim 16, wherein the treatment period is about 1 day to about 21 days.

18. The method of claim 16, wherein the treatment period is about 1 week to about 6 weeks.

19. The method of any one of claims 1-18, wherein the cysteamine precursor compound is administered orally.

20. The method of any one of claims 1-19, wherein the subject is being hospitalized for the betacoronavirus infection.

21. The method of any one of claims 1-20, wherein the subject has a pre-existing condition that places the subject at higher risk of pneumonitis, pneumonia, acute respiratory distress syndrome, respiratory failure, septic shock, organ failure, cytokine storm, or death.

22. The method of claim 21, wherein the pre-existing condition is selected from cardiovascular disease, diabetes, chronic respiratory disease, hypertension, immune deficiency, and obesity.

23. The method of any one of claims 1-22, wherein the subject is at least 40 years old, at least 50 years old, at least 60 years old, at least 70 years old, or at least 80 years old.

24. The method of any one of claims 1-23, wherein the betacoronavirus is SARS-CoV-2.

25. The method of any one of claims 1-23, wherein the betacoronavirus is SARS-CoV-1.

26. The method of any one of claims 1-23, wherein the betacoronavirus is MERS-CoV.

27. The method of any one of claims 1-26, wherein the cysteamine precursor is selected from pantetheine-N-acetyl-L-cysteine disulfide, pantetheine-N-acetylcysteamine disulfide, cysteamine-pantetheine disulfide, cysteamine-4-phosphopantetheine disulfide, cysteamine-gamma-glutamylcysteine disulfide or cysteamine-N-acetylcysteine disulfide, mono-cysteamine-dihydrolipoic acid disulfide, bis-cysteamine-dihydrolipoic acid disulfide, mono-pantetheine-dihydrolipoic acid disulfide, bis-pantetheine-dihydrolipoic acid disulfide, cysteamine-pantetheine-dihydrolipoic acid disulfide, and salts thereof.

28. The method of any one of claims 1-26, wherein the cysteamine precursor is selected from compounds (1)-(3): 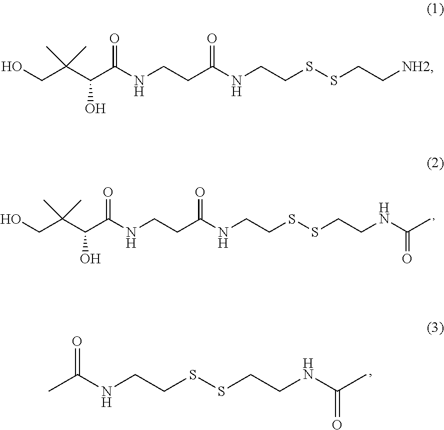
and salts thereof.
